SEQUENCE LISTING (29 sequences)

SEQ ID NO: 1 - protein, 753 aa, Homo sapiens
MAAGSTTLHAVEKLQVRLATKTEPKKLEKYLQKLSALPMTADILAETGIRKTVKRLRKHQHVGDFARDLAARWKKLVLVDRNTRPGPQDPEESASRQRFGEALQDQEKAWGFPENATAPRSPSHSPEHRRTARRTPPGQQRPHPRSHSREPRAERKCPRIAPADSGRYRASPTRTAPLRMPEGPEPAAPGKQPGRGHTHAAQGGPLLCPGCQGQPQGKAVVSHSKGHKSSRQEKRPLCAQGDWHSPTLIREKSCGACLREETPRMPSWASARDRQPSDFKTDKEGGQAGSGQRVPALEEAPDSHQKRPQHSHSNKKRPSLDGRDPGNGTHGLSPEEKEQLSNDRETQEGKPPTAHLDRTSVSSLSEVEEVDMAEEFEQPTLSCEKYLTYDQLRKQKKKTGKSATTALGDKQRKANESKGTRESWDSAKKLPPVQESQSERLQAAGADSAGPKTVPNHVFSELWDLSEAWMQANYDPLSDSDSMTSQAKPEALSSPKFREEAAFPGRRVNAKMPVYSGSRPACQLQVPTLRQQCAQVLRNNPDALSDVGEVPYWVLEPVLEGWRPDQLYRRKKDNHALVRETDELRRNHCFQDFKEEKPQENKTWREQYLRLPDAPEQRLRVMTTNIRSARGNNPNGREAKMICFKSVAKTPYDTSRRQEKSAGDADPENGEIKPASKPAGSSHTPSSQSSSGGGRDSSSSILRWLPEKRANPCLSSSNEHAAPAAKTRKQAAKKVAPLMAKAIRDYKRRFSRR

SEQ ID NO: 2 - protein, 606 aa, Homo sapiens
SWVQMQLEALNLLHTLVWARSLCRAGAVQTQERLSGSASPEQVPAGECCALQEYEAAVEQLKSEQIRAQAEERRKTLSEETRQHQARAQYQDKLARQRYEDQLKQQQLLNEENLRKQEESVQKQEAMRRATVEREMELRHKNEMLRVETEARARAKAERENADIIREQIRLKASEHRQTVLESIRTAGTLFGEGFRAFVTDRDKVTATVAGLTLLAVGVYSAKNATAVTGRFIEARLGKPSLVRETSRITVLEALRHPIQVSRRLLSRPQDVLEGVVLSPSLEARVRDIAIATRNTKKNRGLYRHILLYGPPGTGKTLFAKKLALHSGMDYAIMTGGDVAPMGREGVTAMHKLFDWANTSRRGLLLFMDEADAFLRKRATEEISKDLRATLNAFLYHMGQHSNKFMLVLASNLPEQFDCAINSRIDVMVHFDLPQQEERERLVRLHFDNCVLKPATEGKRRLKLAQFDYGRKCSEVARLTEGMSGREIAQLAVSWQATAYASKDGVLTEAMMDACVQDAVQQYRQKMRWLKAEGPGRGVEHPLSGVQGETLTSWSLATDPSYPCLAGPCTFRICSWMGTGLCPGPLSPRMSCGGGRPFCPPGHPLL

SEQ ID NO: 3 - protein, 325 aa, Homo sapiens
MKPILLQGHERSITQIKYNREGDLLFTVAKDPIVNVWYSVNGERLGTYMGHTGAVWCVDADWDTKHVLTGSADNSCRLWDCETGKQLALLKTNSAVRTCGFDFGGNIIMFSTDKQMGYQCFVSFFDLRDPSQIDNNEPYMKIPCNDSKITSAVWGPLGECIIAGHESGELNQYSAKSGEVLVNVKEHSRQINDIQLSRDMTMFVTASKDNTAKLFDSTTLEHQKTFRTERPVNSAALSPNYDHVVLGGGQEAMDVTTTSTRIGKFEARFFHLAFEEEFGRVKGHFGPINSVAFHPDGKSYSSGGEDGYVRIHYFDPQYFEFEFEA

SEQ ID NO: 4 - protein, 217 aa, Homo sapiens [sequence omitted]

SEQ ID NO: 5 - protein, 523 aa, Homo sapiens
MMLRGNLKQVRIEKNPARLRALESAVGESEPAAAAAMALALAGEPAPPAPAPPEDHPDEEMGFTIDIKSFLKPGEKTYTQRCRLFVGNLPTDITEEDFKRLFERYGEPSEVFINRDRGFGFIRLESRTLAEIAKAELDGTILKSRPLRIRFATHGAALTVKNLSPVVSNELLEQAFSQFGPVEKAVVVVDDRGRATGKGFVEFAAKPPARKALERCGDGAFLLTTTPRPVIVEPMEQFDDEDGLPEKLMQKTQQYHKEREQPPRFAQPGTFEFEYASRWKALDEMEKQQREQVDRNIREAKEKLEAEMEAARHEHQLMLMRQDLMRRQEELRRLEELRNQELQKRKQIQLRHEEEHRRREEEMIRHREQEELRRQQEGFKPNYMENREQEMRMGDMGPRGAINMGDAFSPAPAGNQGPPPMMGMNMNNRATIPGPPMGPGPAMGPEGAANMGTPMMPDNGAVHNDRFPQGPPSQMGSPMGSRTGSETPQAPMSGVGPVSGGPGGFGRGSQGGNFEGPNKRRRY

SEQ ID NO: 6 - protein, 449 aa, Homo sapiens
MMLGTEGGEGFVVKVRGLPWSCSADEVQRFFSDCKIQNGAQGIRFIYTREGRPSGEAFVELESEDEVKLALKKDRETMGHRYVEVFKSNNVEMDWVLKHTGPNSPDTANDGFVRLRGLPFGCSKEEIVQFFSGLEIVPNGITLPVDFQGRSTGEAFVQFASQEIAEKALKKHKERIGHRYIEIFKSSRAEVRTHYDPPRKLMAMQRPGPYDRPGAGRGYNSIGRGAGFERMRRGAYGGGYGGYDDYNGYNDGYGFGSDRFGRDLNYCFSGMSDHRYGDGGSTFQSTTGHCVHMRGLPYRATENDIYNFFSPLNPVRVHIEIGPDGRVTGEADVEFATHEDAVAAMSKDKANMQHRYVELFLNSTAGASGGAYEHRYVELFLNSTAGASGGAYGSQMMGGMGLSNQSSYGGPASQQLSGGYGGGYGGQSSMSGYDQVLQENSSDFQSNIA

SEQ ID NO: 7 - protein, 381 aa, Homo sapiens
MSKGPAVGIDLGTTYSCVGVFQHGKVEIIANDQGNRTTPSYVAFTDTERLIGDAAKNQVAMNPTNTVFDAMRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILS

SEQ ID NO: 8 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTFLIKTVETRDGQVINETSQHHDDLE

SEQ ID NO: 9 - protein, 2442 aa, Homo sapiens
METRSPGLNNMKPQSLQLVLEEQVLALQQQMAENQAASWRKLKNSQEAQQRQATLVRKLQAKVLQYRSWCQELEKRLEATGGPIPQRWENVEEPNLDELLVRLEEEQQRCESLAEVNTQLRLHMEKADVVNKALREDVEKLTVDWSRARDELMRKESQWQMEQEFFKGYLKGEHGRLLSLWREVVTFRRHFLEMKSATDRDLMELKAEHVRLSGSLLTCCLRLTVGAQSREPNGSGRMDGREPAQLLLLLAKTQELEKEAHERSQELIQLKSQGDLEKAELQDRVTELSALLTQSQKQNEDYEKMIKALRETVEILETNHTELMEHEASLSRNAQEEKLSLQQVIKDITQVMVEEGDNIAQGSGHENSLELDSSIFSQFDYQDADKALTLVRSVLTRRRQAVQDLRQQLAGCQEAVNLLQQQHDQWEEEGKALRQRLQKLTGERDTLAGQTVDLQGEVDSLSKERELLQKAREELRQQLEVLEQEAWRLRRVNVELQLQGDSAQGQKEEQQEELHLAVRERERLQEMLMGLEAKQSESLSELITLREALESSHLEGELLRQEQTEVTAALARAEQSIAELSSSENTLKTEVADLRAAAVKLSALNEALALDKVGLNQQLLQLEEENQSVCSRMEAAEQARNALQVDLAEAEKRREALWEKNTHLEAQLQKAEEAGAELQADLRDIQEEKEEIQKKLSESRHQQEAATTQLEQLHQEAKRQEEVLARAVQEKEALVREKAALEVRLQAVERDRQDLAEQLQGLSSAKELLESSLFEAQQQNSVIEVTKGQLEVQIQTVTQAKEVIQGEVRCLKLELDTERSQAEQERDAAARQLAQAEQEGKTALEQQKAAHEKEVNQLREKWEKERSWHQQELAKALESLEREKMELEMRLKEQQTEMEAIQAQREEERTQAESALCQMQLETEKERVSLLETLLQTQKELADASQQLERLRQDMKVQKLKEQETTGILQTQLQEAQRELKEAARQHRDDLAALQEESSSLLQDKMDLQKQVEDLKSQLVAQDDSQRLVEQEVQEKLRETQEYNRIQKELEREKASLTLSLMEKEQRLLVLQEADSIRQQELSALRQDMQEAQGEQKELSAQMELLRQEVKEKEADFLAQEAQLLEELEASHITEQQLRASLWAQEAKAAQLQLRLRSTESQLEALAAEQQPGNQAQAQAQLASLYSALQQALGSVCESRPELSGGGDSAPSVWGLEPDQNGARSLFKRGPLLTALSAEAVASALHKLHQDLWKTQQTRDVLRDQVQKLEERLTDTEAEKSQVHTELQDLQRQLSQNQEEKSKWEGKQNSLESELMELHETMASLQSRLRRAELQRMEAQGERELLQAAKENLTAQVEHLQAAVVEARAQASAAGILEEDLRTARSALKLKNEEVESERERAQALQEQGELKVAQGKALQENLALLTQTLAEREEEVETLRGQIQELEKQREMQKAALELLSLDLKKRNQEVDLQQEQIQELEKCRSVLEHLPMAVQEREQKLTVQREQIRELEKDRETQRNVLEHQLLELEKKDQMIESQRGQVQDLKKQLVTLECLALELEENHHKMECQQKLIKELEGQRETQRVALTHLTLDLEERSQELQAQSSQIHDLESHSTVLARELQERDQEVKSQREQIEELQRQKEHLTQDLERRDQELMLQKERIQVLEDQRTRQTKILEEDLEQIKLSLRERGRELTTQRQLMQERAEEGKGPSKAQRGSLEHMKLILRDKEKEVECQQEHIHELQELKDQLEQQLQGLHRKVGETSLLLSQREQEIVVLQQQLQEAREQGELKEQSLQSQLDEAQRALAQRDQELEALQQEQQQAQGQEERVKEKADALQGALEQAHMTLKERHGELQDHKEQARRLEEELAVEGRRVQALEEVLGDLRAESREQEKALLALQQQCAEQAQEHEVETRALQDSWLQAQAVLKERDQELEALRAESQSSRHQEEAARARAEALQEALGKAHAALQGKEQHLLEQAELSRSLEASTATLQASLDACQAHSRQLEEALRIQEGEIQDQDLRYQEDVQQLQQALAQRDEELRHQQEREQLLEKSLAQRVQENMIQEKQNLGQEREEEEIRGLHQSVRELQLTLAQKEQEILELRETQQRNNLEALPHSHKTSPMEEQSLKLDSLEPRLQRELERLQAALRQTEAREIEWREKAQDLALSLAQTKASVSSLQEVAMFLQASVLERDSEQQRLQDELELTRRALEKERLHSPGATSTAELGSRGEQGVQLGEVSGVEAEPSPDGMEKQSWRQRLEHLQQAVARLEIDRSRLQRHNVQLRSTLEQVERERRKLKREAMRAAQAGSLEISKATASSPTQQDGRGQKNSDAKCVAELQKEVVLLQAQLTLERKQKQDYITRSAQTSRELAGLHHSLSHSLLAVAQAPEATVLEAETRRLDESLTQSLTSPGPVLLHPSPSTTQAASR

SEQ ID NO: 10 - protein, 622 aa, Homo sapiens
MSCRQFSSSYLTSGGGGGGGLGSGGSIRSSYSRFSSSGGRGGGGRFSSSSGYGGGSSRVCGRGGGGSFGYSYGGGSGGGFSASSLGGGFGGGSRGFGGASGGGYSSSGGFGGGFGGGSGGGFGGGYGSGFGGLGGFGGGAGGGDGGILTANEKSTMQELNSRLASYLDKVQALEEANNDLENKIQDWYDKKGPAAIQKNYSPYYNTIDDLKDQIVDLTVGNNKTLLDIDNTRMTLDDFRIKFEMEQNLRQGVDADINGLRQVLDNLTMEKSDLEMQYETLQEELMALKKNHKEEMSQLTGQNSGDVNVEINVAPGKDLTKTLNDMRQEYEQLIAKNRKDIENQYETQITQIEHEVSSSGQEVQSSAKEVTQLRHGVQELEIELQSQLSKKAALEKSLEDTKNRYCGQLQMIQEQISNLEAQITDVRQEIECQNQEYSLLLSIKMRLEKEIETYHNLLEGGQEDFESSGAGKIGLGGRGGSGGSYGRGSRGGSGGSYGGGGSGGGYGGGSGSRGGSGGSYGGGSGSGGGSGGGYGGGSGGGHSGGSGGGHSGGSGGNYGGGSGSGGGSGGGYGGGSGSRGGSGGSHGGGSGFGGESGGSYGGGEEASGSGGGYGGGSGKSSHS

SEQ ID NO: 11 - protein, 91 aa, Homo sapiens
IHTGERPYQCKECGKSFNQSSGLSQHRKIHTLKKPHECDLCGKAFCHRSHLIRHQRIHTGKKPYKCDECGKAFSQSSNLIEHRKTHTGEKP

SEQ ID NO: 12 - protein, 293 aa, Homo sapiens [sequence omitted]

SEQ ID NO: 13 - protein, 1683 aa, Homo sapiens
MPGIKVGGSGVNVNAKGLDLGGRGGVQVPAVDISSSLGGRPVEVQGPSLESGDHAKIKFPTMKVPKFGVSTGREGQTPKAGLRVSAPEVSVGHKGGKPGLTIQAPQLEVSVPSANIEGLEGKLKGPQITGPSLEGDLGLKGAKPQGHIGVDASAPQIGGSITGPSVEVQAPDIDVQGPGSKLNVPKMKVPKFSVSGAKGEETGIDVTLPTGEVTVPGVSGDVSLPEIATGGLEGKMKGTKVKTPEMIIQKPKISMQDVDLSLGSPKLKGDIKVSAPGVQGDVKGPQVALKGSRVDIETPNLEGTLTGPRLGSPSGKTGTCRISMSEVDLNVAAPKVKGGVDVTLPRVEGKVKVPEVDVRGPKVDVSAPDVEAHGPEWNLKMPKMKMPTFSTPGAKGEGPDVHMTLPKGDISISGPKVNVEAPDVNLEGLGGKLKGPDVKLPDMSVKTPKISMPDVDLHVKGTKVKGEYDVTVPKLEGELKGPKVDIDAPDVDVHGPDWHLKMPKMKMPKFSVPGFKAEGPEVDVNLPKADVDISGPKIDVTAPDVSIEEPEGKLKGPKFKMPEMNIKVPKISMPDVDLHLKGPNVKGEYDVTMPKVESEIKVPDVELKSAKMDIDVPDVEVQGPDWHLKMPKMKMPKFSMPGFKAEGPEVDVNLPKADVDISGPKVGVEVPDVNIEGPEGKLKGPKFKMPEMNIKAPKISMPDVDLHMKGPKVKGEYDMTVPKLEGDLKGPKVDVSAPDVEMQGPDWNLKMPKIKMPKFSMPSLKGEGPEFDVNLSKANVDISAPKVDTNAPDLSLEGPEGKLKGPKFKMPEMHFRAPKMSLPDVDLDLKGPKMKGNVDISAPKIEGEMQVPDVDIRGPKVDIKAPDVEGQGLDWSLKIPKMKMPKFSMPSLKGEGPEVDVNLPKADVDVSGPKVDIEAPDVSLEGPEGKLKGPKFKMPEMHFKTPKISMPDVDLHLKGPKVKGDVDVSVPKVEGEMKVPDVEIKGPKMDIDAPDVEVQGPDWHLKMPKMKMPKFSMPGFKGEGREVDVNLPKADIDVSGPKVDVEVPDVSLEGPEGKLKGPKFKMPEMHFKAPKISMPDVDLNLKGPKLKGDVDVSLPEVEGEMKVPDVDIKGPKVDISAPDVDVHGPDWHLKMPKVKMPKFSMPGFKGEGPEVDVKLPKADVDVSGPKMDAEVPDVNIEGPDAKLKGPKFKMPEMSIKPQKISIPDVGLHLKGPKMKGDYDVTVPKVEGEIKAPDVDIKGPKVDINAPDVEVHGPDWHLKMPKVKMPKFSMPGFKGEGPEVDMNLPKADLGVSGPKVDIDVPDVNLEAPEGKLKGPKFKMPSMNIQTHKISMPDVGLNLKAPKLKTDVDVSLPKVEGDLKGPEIDVKAPKMDVNVGDIDIEGPEGKLKGPKFKMPEMHFKAPKISMPDVDLHLKGPKVKGDMDVSVPKVEGEMKVPDVDIKGPKVDIDAPDVEVHDPDWHLKMPKMKMPKFSMPGFKAEGPEVDVNLRKADIDVSGPSVDTDAPDLDIEGPEGKLKGSKFKMPKLNIKAPKVSMPDVDLNLKGPKLKGEIDASVPELEGDLRGPQVDVKGPLVEAEVPDVDLECPDAKLKGPKFKMPEMHFKAPKISMPDVDLHLKGPKVKGDADVSVPKLEGDLTGPSVGVEVPDVELECPDAKLKGPKFKMPDMHFKAPKISMPDVDLH

SEQ ID NO: 14 - protein, 284 aa, Homo sapiens
MEAIKKKMQMLKLDKENALDRAEQAEAEQKQAEERSKQLEDELAAMQKKLKGTEDELDKYSEALKDAQEKLELAEKKAADAEAEVASLNRRIQLVEEELDRAQERLATALQKLEEAEKAADESERGMKVIENRALKDEEKMELQEIQLKEAKHIAEEADRKYEEVARKLVIIEGDLERTEERAELAESKCSELEEELKNVTNNLKSLEAQAEKYSQKEDKYEEEIKILTDKLKEAETRAEFAERSVAKLEKTIDDLEDELYAQKLKYKAISEELDHALNDMTSI

SEQ ID NO: 15 - protein, 529 aa, Homo sapiens
MLGFVGRVAAAPASGALRRLTPSASLPPAQLLLRAAPTAVHPVRDYAAQTSPSPKAGAATGRIVAVIGAVVDVQFDEGLPPILNALEVQGRETRLVLEVAQHLGESTVRTIAMDGTEGLVRGQKVLDSGAPIKIPVGPETLGRIMNVIGEPIDERGPIKTKQFAPIHAEAPEFMEMSVEQEILVTGIKVVDLLAPYAKGGKIGLFGGAGVGKTVLIMELINNVAKAHGGYSVFAGVGERTREGNDLYHEMIESGVINLKDATSKVALVYGQMNEPPGARARVALTGLTVAEYFRDQEGQDVLLFIDNIFRFTQAGSEVSALLGRIPSAVGYQPTLATDMGTMQERITTTKKGSITSVQAIYVPADDLTDPAPATTFAHLDATTVLSRAIAELGIYPAVDPLDSTSRIMDPNIVGSEHYDVARGVQKILQDYKSLQDIIAILGMDELSEEDKLTVSRARKIQRFLSQPFQVAEVFTGHMGKLVPLKETIKGFQQILAGEYDHLPEQAFYMVGPIEEAVAKADKLAEEHSS

SEQ ID NO: 16 - protein, 184 aa, Homo sapiens
PGLTNKTSQLKSSSINQSTSNKKISQYRIRLEEKQKLRFHYGIIERQLLNYVRIARKAKGSTGEVLLQLLEMRSDNVIFRLGMAPTIPGARQLVNHRHILVNDRIVDIPSYRCKPQDFITIKNRQKSQAIINKNIDFYQKYKIPNHLTYSSLEKKGFVNQILDRESIGLKINELLVVEYYSRQA

SEQ ID NO: 17 - protein, 6669 aa, Homo sapiens
MADDEDYEEVVEYYTEEVVYEEVPGETITKIYETTTTRTSDYEQSETSKPALAQPALAQPASAKPVERRKVIRKKVDPSKFMTPYIAHSQKMQDLFSPNKYKEKFEKTKGQPYASTTDTPELRRIKKVQDQLSEVKYRMDGDVAKTICHVDEKAKDIEHAKKVSQQVSKVLYKQNWEDTKDKYLLPPDAPELVQAVKNTAMFSKKLYTEDWEADKSLFYPYNDSPELRRVAQAQKALSDVAYKKGLAEQQAQFTPLADPPDIEFAKKVTNQVSKQKYKEDYENKIKGKWSETPCFEVANARMNADNISTRKYQEDFENMKDQIYFMQTETPEYKMNKKAGVAASKVKYKEDYEKNKGKADYNVLPASENPQLRQLKAAGDALSDKLYKENYEKTKAKSINYCETPKFKLDTVLQNFSSDKKYKDSYLKDILGHYVGSFEDPYHSHCMKVTAQNSDKNYKAEYEEDRGKGFFPQTITQEYEAIKKLDQCKDHTYKVHPDKTKFTQVTDSPVLLQAQVNSKQLSDLNYKAKHESEKFKCHIPPDTPAFIQHKVNAYNLSDNLYKQDWEKSKAKKFDIKVDAIPLLAAKANTKNTSDVMYKKDYEKNKGKMIGVLSINDDPKMLHSLKVAKNQSDRLYKENYEKTKAKSMNYCETPKYQLDTQLKNFSEARYKDLYVKDVLGHYVGSMEDPYHTHCMKVAAQNSDKSYKAEYEEDKGKCYFPQTITQEYDAIKKLDQCKDHTYKVHPDKTKFTAVTDSPVLLQAQLNTKQLSDLNYKAKHEGERFKCHIPADAPQFIQHRVNAYNLSDNVYKQDWEKSKAKKFDIKVDAIPLLAAKANTKNTSDVMYKKDYEKSKGKMIGALSINDDPKMLHSLKTAKNQSDREYRKDYEKSKTIYTAPLDMLQVTQAKKSQAIASDVDYKHILHSYSYPPDSINVDLAKKAYALQSDVEYKADYNSWMKGCGWVPFGSLEMEKAKRASDILNEKKYRQHPDTLKFTSIEDAPITVQSKINQAQRSDIAYKAKGEEIIHNYNLPPDLPQFIQAKVNAYNISENMYKADLKDLSKKGYDLRTDAIPIRAAKAARQAASDVQYKKDYEKAKGKMVGFQSLQDDPKLVHYMNVAKIQSDREYKKDYEKTKSKYNTPHDMFNVVAAKKAQDVVSNVNYKHSLHHYTYLPDAMDLELSKNMMQIQSDNVYKEDYNNWMKGIGWIPIGSLDVEKVKKAGDALNEKKYRQHPDTLKFTSIVDSPVMVQAKQNTKQVSDILYKAKGEDVKHKYTMSPDLPQFLQAKCNAYSISDVCYKRDWHDLIRKGNNVLGDAIPITAAKASRNIASDYKYKEAYEKSKGKHVGFRSLQDDPKLVHYMNVAKLQSDREYKKNYENTKTSYHTPGDMVTITAAKMAQDVATNVNYKQPLHHYTYLPDAMSLEHTRNVNQIQSDNVYKDEYNSFLKGIGWIPIGSLEVEKVKKAGDALNERKYRQHPDTVKFTSVPDSMGMMLAQHNTKQLSDLNYKVEGEKLKHKYTIDPELPQFIQAKVNALNMSDAHYKADWKKTIRKGYDLRPDAIPIVAAKSSRNIASDCKYKEAYEKAKGKQVGFLSLQDDPKLVHYMNVAKIQSDREYKKGYEASKTKYHTPLDMVSVTAAKKSQEVATNANYRQSYHHYTLLPDALNVEHSRNAMQIQSDNLYKSDFTNWMKGIGWVPIESLEVEKAKKAGEILSEKKYRQHPEKLKFTYAMDTMEQALNKSNKLNMDKRLYTEKWNKDKTTIHVMPDTPDILLSRVNQITMSDKLYKAGWEEEKKKGYDLRPDAIAIKAARASRDIASDYKYKKAYEQAKGKHIGFRSLEDDPKLVHFMQVAKMQSDREYKKGYEKSKTSFHTPVDMLSVVAAKKSQEVATNANYRNVIHTYNMLPDAMSFELAKNMMQIQSDNQYKADYADFMKGIGWLPLGSLEAEKNKKAMEIISEKKYRQHPDTLKYSTLMDSMNMVLAQNNAKIMNEHLYKQAWEADKTKVHIMPDIPQIILAKANAINISDKLYKLSLEESKKKGYDLRPDAIPIKAAKASRDIASDYKYKYNYEKGKGKMVGFRSLEDDPKLVHSMQVAKMQSDREYKKNYENTKTSYHTPADMLSVTAAKDAQANITNTNYKHLIHKYILLPDAMNIELTRNMNRIQSDNEYKQDYNEWYKGLGWSPAGSLEVEKAKKATEYASDQKYRQHPSNFQFKKLTDSMDMVLAKQNAHTMNKHLYTIDWNKDKTKIHVMPDTPDILQAKQNQTLYSQKLYKLGWEEALKKGYDLPVDAISVQLAKASRDIASDYKYKQGYRKQLGHHVGFRSLQDDPKLVLSMNVAKMQSEREYKKDFEKWKTKFSSPVDMLGVVLAKKCQELVSDVDYKNYLHQWTCLPDQNDVVQAKKVYELQSENLYKSDLEWLRGIGWSPLGSLEAEKNKRASEIISEKKYRQPPDRNKFTSIPDAMDIVLAKTNAKNRSDRLYREAWDKDKTQIHIMPDTPDIVLAKANLINTSDKLYRMGYEELKRKGYDLPVDAIPIKAAKASREIASEYKYKEGFRKQLGHHIGARNIEDDPKMMWSMHVAKIQSDREYKKDFEKWKTKFSSPVDMLGVVLAYKCQTLVSDVDYKNYLHQWTCLPDQSDVIHARQAYDLQSDNLYKSDLQWLKGIGWMTSGSLEDEKNKRATQILSDHVYRQHPDQFKFSSLMDSIPMVLAKNNAITMNHRLYTEAWDKDKTTVHIMPDTPEVLLAKQNKVNYSEKLYKLGLEEAKRKGYDMRVDAIPIKAAKASRDIASEFKYKEGYRKQLGHHIGARAIRDDPKMMWSMHVAKIQSDREYKKDFEKWKTKFSSPVDMLGVVLAKKCQTLVSDVDYKNYLHQWTCLPDQSDVIHARQAYDLQSDNMYKSDLQWMRGIGWVSIGSLDVEKCKRATEILSDKIYRQPPDRFKFTSVTDSLEQVLAKNNALNMNKRLYTEAWDKDKTQIHIMPDTPEIMLARQNKINYSETLYKLANEEAKKKGYDLRSDAIPIVAAKASRDVISDYKYKDGYRKQLGHHIGARNIEDDPKMMWSMHVAKIQSDREYKKDFEKWKTKFSSPVDMLGVVLAKKCQTLVSDVDYKNYLHEWTCLPDQNDVIHARQAYDLQSDNIYKSDLQWLRGIGWVPIGSMDVVKCKRAAEILSDNIYRQPPDKLKFTSVTDSLEQVLAKNNALNMNKRLYTEAWDKDKTQVHIMPDTPEIMLARQNKINYSESLYRQAMEEAKKEGYDLRSDAIPIVAAKASRDIASDYKYKEAYRKQLGHHIGARAVHDDPKIMWSLHIAKVQSDREYKKDFEKYKTRYSSPVDMLGIVLAKKCQTLVSDVDYKHPLHECICLPDQNDIIHARKAYDLQSDNLYKSDLEWMKGIGWVPIDSLEVVRAKRAGELLSDTIYRQRPETLKFTSITDTPEQVLAKNNALNMNKRLYTEAWDNDKKTIHVMPDTPEIMLAKLNRINYSDKLYKLALEESKKEGYDLRLDAIPIQAAKASRDIASDYKYKEGYRKQLGHHIGARNIKDDPKMMWSIHVAKIQSDREYKKEFEKWKTKFSSPVDMLGVVLAKKCQILVSDIDYKHPLHEWTCLPDQNDVIQARKAYDLQSDAIYKSDLEWLRGIGWVPIGSVEVEKVKRAGEILSDRKYRQPADQLKFTCITDTPEIVLAKNNALTMSKHLYTEAWDADKTSIHVMPDTPDILLAKSNSANISQKLYTKGWDESKMKDYDLRADAISIKSAKASRDIASDYKYKEAYEKQKGHHIGAQSIEDDPKIMCAIHAEKIQSEREYKKEFQKWKTKFSSPVDMLSILLAKKCQTLVTDIYYRNYLHEWTCMPDQNDIIQAKKAYDLQSDALYKADLEWLRGIGWMPQGSPEVLRVKNAQNIFCDSVYRTPVVNLKYTSIVDTPEVVLAKSNAENISIPKYREVWDKDKTSIHIMPDTPEINLARANALNVSNKLYREGWDEMKAGCDVRLDAIPIQAAKASREIASDYKYKLDHEKQKGHYVGTLTARDDNKIRWALIADKLQNEREYRLDWAKWKAKIQSPVDMLSILHSKNSQALVSDMDYRNYLHQWTCMPDQNDVIQAKKAYELQSDNVYKADLEWLRGIGWMPNDSVSVNHAKHAADIFSEKKYRTKIETLNFTPVDDRVDYVTAKQSGEILDDIKYRKDWNATKSKYTLTETPLLHTAQEAARILDQYLYKEGWERQKATGYILPPDAVPFVHAHHCNDVQSELKYKAEHVKQKGHYVGVPTMRDDPKLVWFEHAGQIQNERLYKEDYHKTKAKINIPADMVSVLAAKQGQTLVSDIDYRNYLHQWMCHPDQNDVIQARKAYDLQSDNVYRADLEWLRGIGWIPLDSVDHVRVTKNQEMMSQIKYKKNALENYPNFTSVVDPPEIVLAKINSVNQSDVKYKETFNKAKGKYTFSPDTPHISHSKDMGKLYSTILYKGAWEGTKAYGYTLDERYIPIVGAKHADLVNSELKYKETYEKQKGHYLAGKVIGEFPGVVHCLDFQKMRSALNYRKHYEDTKANVHIPNDMMNHVLAKRCQYILSDLEYRHYFHQWTSLLEEPNVIRVRNAQEILSDNVYKDDLNWLKGIGCYVWDTPQILHAKKSYDLQSQLQYTAAGKENLQNYNLVTDTPLYVTAVQSGINASEVKYKENYHQIKDKYTTVLETVDYDRTRNLKNLYSSNLYKEAWDRVKATSYILPSSTLSLTHAKNQKHLASHIKYREEYEKFKALYTLPRSVDDDPNTARCLRVGKLNIDRLYRSVYEKNKMKIHIVPDMVEMVTAKDSQKKVSEIDYRLRLHEWICHPDLQVNDHVRKVTDQISDIVYKDDLNWLKGIGCYVWDTPEILHAKHAYDLRDDIKYKAHMLKTRNDYKLVTDTPVYVQAVKSGKQLSDAVYHYDYVHSVRGKVAPTTKTVDLDRALHAYKLQSSNLYKTSLRTLPTGYRLPGDTPHFKHIKDTRYMSSYFKYKEAYEHTKAYGYTLGPKDVPFVHVRRVNNVTSERLYRELYHKLKDKIHTTPDPPEIRQVKKTQEAVSELIYKSDFFKMQGHMISLPYTPQVIHCRYVGDITSDIKYKEDLQVLKGFGCFLYDTPDMVRSRHLRKLWSNYLYTDKAREMRDKYKVVLDTPEYRKVQELKTHLSELVYRAAGKKQKSIFTSVPDTPDLLRAKRGQKLQSQYLYVELATKERPHHHAGNQTTALKHAKDVKDMVSEKKYKIQYEKMKDKYTPVPDTPILIRAKRAYWNASDLRYKETFQKTKGKYHTVKDALDIVYHRKVTDDISKIKYKENYMSQLGIWRSIPDRPEHFHHRAVTDTVSDVKYKEDLTWLKGIGCYAYDTPDFTLAEKNKTLYSKYKYKEVFERTKSDFKYVADSPINRHFKYATQLMNEKKYRADYEQRKDKYHLVVDEPRHLLAKTRSDQISQIKYRKNYEKSKDKFTSIVDTPEHLRTTKVNKQISDILYKLEYNKAKPRGYTTIHDTPMLLHVRKVKDEVSDLKYKEVYQRNKSNCTIEPDAVHIKAAKDAYKVNTNLDYKKQYEANKAHWKWTPDRPDFLQAAKSSLQQSDFEYKLDREFLKGCKLSVTDDKNTVLALRNTLIESDLKYKEKHVKERGTCHAVPDTPQILLAKTVSNLVSENKYKDHVKKHLAQGSYTTLPETRDTVHVKEVTKHVSDTNYKKKFVKEKGKSNYSIMLEPPEVKHAMEVAKKQSDVAYRKDAKENLHYTTVADRPDIKKATQAAKQASEVEYRAKHRKEGSHGLSMLGRPDIEMAKKAAKLSSQVKYRENFDKEKGKTPKYNPKDSQLYKVMKDANNLASEVKYKADLKKLHKPVTDMKESLIMNHVLNTSQLASSYQYKKKYEKSKGHYHTIPDNLEQLHLKEATELQSIVKYKEKYEKERGKPMLDFETPTYITAKESQQMQSGKEYRKDYEESIKGRNLTGLEVTPALLHVKYATKIASEKEYRKDLEESIRGKGLTEMEDTPDMLRAKNATQILNEKEYKRDLELEVKGRGLNAMANETPDFMRARNATDIASQIKYKQSAEMEKANFTSVVDTPEIIHAQQVKNLSSQKKYKEDAEKSMSYYETVLDTPEIQRVRENQKNFSLLQYQCDLKNSKGKITVVQDTPEILRVKENQKNFSSVLYKEDVSPGTAIGKTPEMMRVKQTQDHISSVKYKEAIGQGTPIPDLPEVKRVKETQKHISSVMYKENLGTGIPTTVTPEIERVKRNQENFSSVLYKENLGKGIPTPITPEMERVKRNQENFSSVLYKENMGKGTPLPVTPEMERVKHNQENISSVLYKENVGKATATPVTPEMQRVKRNQENISSVLYKENLGKATPTPFTPEMERVKRNQENFSSVLYKENMRKATPTPVTPEMERAKRNQENISSVLYSDSFRKQIQGKAAYVLDTPEMRRVRETQRHISTVKYHEDFEKHKGCFTPVVTDPITERVKKNMQDFSDINYRGIQRKVVEMEQKRNDQDQETITGLRVWRTNPGSVFDYDPAEDNIQSRSLHMINVQAQRRSREQSRSASALSVSGGEEKSEHSEAPDHHLSTYSDGGVFAVSTAYKHAKTTELPQQRSSSVATQQTTVSSIPSHPSTAGKIFRAMYDYMAADADEVSFKDGDAIINVQAIDEGWMYGTVQRTGRTGMLPANYVEAI

SEQ ID NO: 18 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 19 - protein, 705 aa, Homo sapiens
MWLLYLLVPALFCRAGGSIPIPQKLFGEVTSPLFPKPYPNNFETTTVITVPTGYRVKLVFQQFDLEPSEGCFYDYVKISADKKSLGRFCGQLGSPLGNPPGKKEFMSQGNKMLLTFHTDFSNEENGTIMFYKGFLAYYQAVDLDECASRSKSGEEDPQPQCQHLCHNYVGGYFCSCRPGYELQEDRHSCQAECSSELYTEASGYISSLEYPRSYPPDLRCNYSIRVERGLTLHLKFLEPFDIDDHQQVHCPYDQLQIYANGKNIGEFCGKQRPPDLDTSSNAVDLLFFTDESGDSRGWKLRYTTEIIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 20 - protein, 704 aa, Homo sapiens
VLGGLLLLGVAQLGLFLEEKLREHLAEKEKLNEERLEQEEKLKAKIRQLTEEKAALEEYITQERNRAKETLEEERKRMQELESLLAQQKKALAKSITQEKNRVKEALEEEQTRVQELEERLARQKEISESNIAYEKRKAKEAMEKEKKKVQDLENRLTKQKEELELKEQKEDVLNNKLSDALAMVEETQKTKATESLKAESLALKLNETLAELETTKTKMIMVEERLILQQKMVKALQDEQESQRHGFGEEIMEYKEQIKQHAQTIVSLEEKLQKVTQHHKKIEGEIATLKDNDPAPKEERPQDPLVAPMTESSAKDMAYEHLIDDLLAAQKEILSQQEVIMKLRKDLTEAHSRMSDLRGELNEKQKMELEQNVVLVQQQSKELSVLKEKMAQMSSLVEKKDRELKALEEALRASQEKHRLQLNTEKEQKPRKKTQTCDTSVQIEPVHTEAFSSSQEQQSFSDLGVRCKGSRHEEVIQRQKKALSELRARIKELEKARSPDHKDHQNESFLDLKNLRMENNVQKILLDAKPDLPTLSRIEILAPQNGLCNARFGSAMEKSGKMDVAEALELSEKLYLDMSKTLGSLMNIKNMSGHVSMKYLSRQEREKVNQLRQRDLDLVFDKITQLKNQLGRKEELLRGYEKDVEQLRRSKVSIEMYQSQVAKLEDDIYKEAEEKALLKEALERMEHQLCQEKRINRAIRQQK

SEQ ID NO: 21 - protein, 1956 aa, Homo sapiens
LYLPHLTKKIMVESCLLTFRAFFWWIALIKMDLSDLGEAAAFLRRSEAELLLLQATALDGKKKCWIPDGENAYIEAEVKGSEDDGTVIVETADGESLSIKEDKIQQMNPPEFEMIEDMAMLTHLNEASVLHTLKRRYGQWMIYTYSGLFCVTINPYKWLPVYQKEVMAAYKGKRRSEAPPHIFAVANNAFQDMLHNRENQSILFTGESGAGKTVNSKHIIQYFATIAAMIESRKKQGALEDQIMQANTILEAFGNAKTLRNDNSSRFGKFIRMHFGARGMLSSVDIDIYLLEKSRVIFQQAGERNYHIFYQILSGQKELHDLLLVSANPSDFHFCSCGAVTVESLDDAEELLATEQAMDILGFLPDEKYGCYKLTGAIMHFGNMKFKQKPREEQLEADGTENADKAAFLMGINSSELVKCLIHPRIKVGNEYVTRGQTIEQVTCAVGALSKSMYERMFKWLVARINRALDAKLSRQFFIGILDITGFEILEYNSLEQLCINFTNEKLQQFFNWYMFVLEQEEYKKESIEWVSIGFGLDLQACIDLIEKPMGILSILEEECMFPKATDLTFKTKLFDNHFGKSVHLQKPKPDKKKFEAHFELVHYAGVVPYNISGWLEKNKDLLNETVVAVFQKSSNRLLASLFENYMSTDSAIPFGEKKRKKGASFQTVASLHKENLNKLMTNLKSTAPHFVRCINPNVNKIPGILDPYLVLQQLRCNGVLEGTRICREGFPNRLQYADFKQRYCILNPRTFPKSKFVSSRKAAEELLGSLEIDHTQYRFGITKVFFKAGFLGQLEAIRDERLSKVFTLFQARAQGKLMRIKFQKILEERDALILIQWNIRAFMAVKNWPWMRLFFKIKPLVKSSEVGEEVAGLKEECAQLQKALEKSEFQREELKAKQVSLTQEKNDLILQLQAEQETLANVEEQCEWLIKSKIQLEARVKELSERVEEEEEINSELTARGRKLEDECFELKKEIDDLETMLVKSEKEKRTTEHKVKNLTEEVEFLNEDISKLNRAAKVVQEAHQQTLDDLHMEEEKLSSLSKANLKLEQQVDELEGALEQERKARMNCERELHKLEGNLKLNRESMENLESSQRHLAEELRKKELELSQMNSKVENEKGLVAQLQKTVKELQTQIKDLKEKLEAERTTRAKMERERADLTQDLADLNERLEEVGGSSLAQLEITKKQETKIQKLHRDMEEATLHFETTSASLKKRHADSLAELEGQVENLQQVKQKLEKDKSDLQLEVDDLLTRVEQMTRAKANAEKLCTLYEERLHEATAKLDKVTQLANDLAAQKTKLWSESGEFLRRLEEKEALINQLSREKSNFTRQIEDLRGQLEKETKSQSALAHALQKAQRDCDLLREQYEEEQEVKAELHRTLSKVNAEMVQWRMKYENNVIQRTEDLEDAKKELAIRLQEAAEAMGVANARNASLERARHQLQLELGDALSDLGKVRSAAARLDQKQLQSGKALADWKQKHEESQALLDASQKEVQALSTELLKLKNTYEESIVGQETLRRENKNLQEEISNLTNQVREGTKNLTEMEKVKKLIEEEKTEVQVTLEETEGALERNESKILHFQLELLEAKAELERKLSEKDEEIENFRRKQQCTIDSLQSSLDSEAKSRIEVTRLKKKMEEDLNEMELQLSCANRQVSEATKSLGQLQIQIKDLQMQLDDSTQLNSDLKEQVAVAERRNSLLQSELEDLRSLQEQTERGRRLSEEELLEATERINLFYTQNTSLLSQKKKLEADVARMQKEAEEVVQECQNAEEKAKKAAIEAANLSEELKKKQDTIAHLERTRENMEQTITDLQKRLAEAEQMALMGSRKQIQKLESRVRELEGELEGEIRRSAEAQRGARRLERCIKELTYQAEEDKKNLSRMQTQMDKLQLKVQNYKQQVEVAETQANQYLSKYKKQQHELNEVKERAEVAESQVNKLKIKAREFGKKVQEE

SEQ ID NO: 22 - protein, 1339 aa, Homo sapiens
KTALLLHGVFLLLLIFFLEQGKGNLHVTSPEDAECRRTKERLSNGNSRGSVSKSSRNIPRRHTLGGPRSSKEILGMQTSEMDRKREAFLEHLKQKYPHHASAIMGHQERLRDQTRSPKLSHSPQPPSLGDPVEHLSETSADSLEAMSEGDAPTPFSRGSRTRASLPVVRSTNQTKERSLGVLYLQYGDETKQLRMPNEITSADTIRALFVSAFPQQLTMKMLESPSVAIYIKDESRNVYYELNDVRNIQDRSLLKVYNKDPAHAFNHTPKTMNGDMRMQRELVYARGDGPGAPRPGSTAHPPHAIPNSPPSTPVPHSMPPSPSRIPYGGTRSMVVPGNATIPRDRISSLPVSRPISPSPSAILERRDVKPDEDMSGKNIAMYRNEGFYADPYLYHEGRMSIASSHGGHPLDVPDHIIAYHRTAIRSASAYCNPSMQAEMHMEQSLYRQKSRKYPDSHLPTLGSKTPPASPHRVSDLRMIDMHAHYNAHGPPHTMQPDRASPSRQAFKKEPGTLVYIEKPRSAAGLSSLVDLGPPLMEKQVFAYSTATIPKDRETRERMQAMEKQIASLTGLVQSALFKGPITSYSKDASSEKMMKTTANRNHTDSAGTPHVSGGKMLSALESTVPPSQPPPVGTSAIHMSLLEMRRSVAELRLQLQQMRQLQLQNQELLRAMMKKAELEISGKVMETMKRLEDPVQRQRVLVEQERQKYLHEEEKIVKKLCELEDFVEDLKKDSTAASRLVTLKDVEDGAFLLRQVGEAVATLKGEFPTLQNKMRAILRIEVEAVRFLKEEPHKLDSLLKRVRSMTDVLTMLRRHVTDGLLKGTDAAQAAQYMAMEKATAAEVLKSQEEAAHTSGQPFHSTGAPGDAKSEVVPLSGMMVRHAQSSPVVIQPSQHSVALLNPAQNLPHVASSPAVPQEATSTLQMSQAPQSPQIPMNGSAMQSLFIEEIHSVSAKNRAVSIEKAEKKWEEKRQNLDHYNGKEFEKLLEEAQANIMKSIPNLEMPPATGPLPRGDAPVDKVELSEDSPNSEQDLEKLGGKSPPPPPPPPRRSYLPGSGLTTTRSGDVVYTGRKENITAKASSEDAGPSPQTRATKYPAEEPASAWTPSPPPVTTSSSKDEEEEEEEGDKIMAELQGSSGAPQTSRMPVPMSAKNRPGTLDKPGKQSKLQDPRQYRQANGSAKKSGGDFKPTSPSLPASKIPALSPSSGKSSSLPSSSGDSSNLPNPPATKPSIASNPLSPQTGPPAHSASLIPSVSNGSLKFQSLTHTGKGHHLSFSPQSQNGRAPPPLSFSSSPPSPASSVSLNQGAKGTRTIHTPSLTSYKAQNGSSSKATPSTAKETS

SEQ ID NO: 23 - protein, 520 aa, Homo sapiens
MTQNYEGQLQSLKAQFSKLTNNFEKLRLHQMKQNKVPRKELPHLKEEIPFELSNLNQKLEEFRAKSREWDKQEILYQTHLISLDAQQKLLSEKCNQFQKQAQSYQTQLNGKKQCLEDSSSEIPRLICDPDPNCEINERDEFIIEKLKSAVNEIALSRNKLQDENQKLLQELKMYQRQCQAMEAGLSEVKSELQSRDDLLRIIEMERLQLHRELLKIGECQNAQGNKTRLESSYLPSIKEPERKIKELFSVMQDQPNHEKELNKIRSQLQQVEEYHNSEQERMRNEISDLTEELHQKEITIATVTKKAALLEKQLKMELEIKEKMLAKQKVSDMKYKAVRTENTHLKGMMGDLDPGRYMSMDFTNREQSRHTSINKLQYENERLRNDLAKLHVNGKSTWTNQNTYEETGRYAYQSQIKVEKNEERLSHDCEPNRSTMPPLPPSTFQAKEMTSPLVSDDDVFPLSPPDMSFPASLAAQHFLLEEEKRAKELEKLLNTHIDELQRHTEFTLNKYSKLKQNRHI

SEQ ID NO: 24 - protein, 452 aa, Homo sapiens
HEGPRRRGRLWDSSGVPQRQKRPGPWRTQTQEQMSRDVCIHTWPCTYYLEPKRRWVTGQLSLTSLSLRFMTDSTGEILVSFPLSSIVEIKKEASHFIFSSITILEKGHAKHWFSSLRPSRNVVFSIIEHFWRELLLSQPGAMADASVPRTRGEELTGLMAGSQKRLEDTARVLHHQGQQLDSVMRGLDKMESDLEVADRLLTELESPAWWPFSSKLWKTPPETKPREDVSMTSCEPFGKEGILIKIPAVISHRTESHVKPGRLTVLVSGLEIHDSSSLLMHRFEREDVDDIKVHSPYEISIRQRFIGKPDMAYRLISAKMPEVIPILEVQFSKKMELLEDALVLRSARTSSPAEKSCSVWHAASGLMGRTLHREPPAGDQEGTALHLQTSLPALSEADTQELTQILRRMKGLALEAESELERQDEALDGVAAAVDRATLTIDKHNRRMKRLT

SEQ ID NO: 25 - protein, 35 aa, Homo sapiens
MVLGLTEMLRSCLLYALRPHCCHCHRMPFSIRGHL

SEQ ID NO: 26 - protein, 146 aa, Homo sapiens [sequence omitted]

SEQ ID NO: 27 - protein, 140 aa, Homo sapiens
MAGWQSYVDNLMCDGCCQEAAIVGYCDAKYVWAATAGGVFQSITPIEIDMIVGKDREGFFTNGLTLGAKKCSVIRDSLYVDGDCTMDIRTKSQGGEPTYNVAVGRAGRVLVFVMGKEGVHGGGLNKKAYSMAKYLRDSGF

SEQ ID NO: 28 - DNA, 37 bp, Artificial sequence
  PCR primer used in conjunction with SEQ ID NO 29 to amplify the KIAA1273/TOB3 cDNA
cgatggtaccgatcctgggtgcagatgcagctggaag

SEQ ID NO: 29 - DNA, 34 bp, Artificial sequence
  PCR primer used in conjunction with SEQ ID NO 28 to amplify the KIAA1273/TOB3 cDNA
atcgctcgagctacaacagggggtgccctggggg

(19) United States
(12) Patent Application Publication
(10) Pub. No.: US 2007/0009501 A1
(43) Pub. Date: Jan. 11, 2007

(54) Squamous cell carcinoma antigens and use therefor

(76) Inventors: Oliver Gires, Muenchen (DE); Reinhard Zeidler, Olching (DE); Markus Muenz, Greiling (DE); Martina Schaffrik, Muenchen (DE)

Correspondence Address: JENKINS, WILSON, TAYLOR & HUNT, P. A., 3100 TOWER BLVD, SUITE 1200, DURHAM, NC, 27707, US

(21) Appl. No.: 11/471,740

(22) Filed: Jun. 21, 2006

Related U.S. Application Data
(63) Continuation of application No. PCT/EP04/14634, filed on Dec. 22, 2004.

(30) Foreign Application Priority Data
Dec. 22, 2003 (DE) 10360456.1

(51) Int. Cl. A61K 48/00; C12N 5/08

(52) U.S. Cl. 424/93.21; 424/93.7; 435/372

(57) ABSTRACT

The present invention relates to tumor antigens of the squamous epithelial carcinoma, nucleic acids encoding the same as well as antibodies directed against the same. The invention furthermore relates to methods for the generation of antigen presenting cells and T cells specific for such antigens. Eventually, the invention comprises diagnostic and therapeutic methods for the detection/for the treatment of a squamous epithelial carcinoma, in particular a squamous epithelial carcinoma in the otolaryngologic, head, and neck region.

### RELATED APPLICATIONS

This application is a continuation of PCT International Patent Application No. PCT/EP2004/014634, filed Dec. 22, 2004, which claims priority to German Patent Application No. 10360456.1, filed Dec. 22, 2003, the disclosures of each of which are incorporated herein by reference in their entitety.

The present invention relates to tumor antigens of the squamous epithelial carcinoma, nucleic acids encoding the same as well as antibodies directed against the same. The invention furthermore relates to methods for the generation of antigen-presenting cells and T cells specific for these antigens. Eventually, the invention comprises diagnostic and therapeutic methods for the detection and treatment, respectively, of a squamous epithelial carcinoma, in particular of a squamous epithelial carcinoma of the otolaryngologic, head, and neck region.

Carcinoma cells differ in their expression pattern from healthy epithelium both on the RNA and on the protein level. The overexpression of specific proteins or the formation of mutant or post-translationally altered proteins in tumor cells can lead to an immune response. This is accompanied by the activation of specific T and B lymphocytes and the formation of specific antibodies.

Such tumor-specific antibodies are in principle suitable as diagnostic markers for the detection of a tumor disease. Since, in addition, such antibodies can be present already in early tumor stages in the serum of patients they are similarly suitable as biomarkers for the early recognition of cancer.

Because a human cell can possibly form up to 100.000 and more different proteins, tumor-specific antibodies from the serum of cancer patients are a suitable means to identify immunogenic tumor-associated antigens.

A technology has been developed (AMIDA for autoantibody-mediated identification of antigens) which utilises such antibodies to isolate by means of immunoprecipitation and subsequent two-dimensional (2D) gel electrophoresis from the lysate of tumor cells exclusively those proteins against which specific antibodies exist. This technology is described in the PCT application No. WO 03/025568 the contents of which is included herein by reference in its entirety.

It is known that an individual set of autoantibodies is present in every human the generation and importance of which is unclear. Usually these antibodies do not cause any symptoms. To be able to distinguish proteins recognized by these ‘natural’ autoantibodies from tumor-specific proteins the inventors carried out AMIDA for control purposes also with immunoglobulins derived from non-tumor patients. In this respect two different methods can be distinguished:
1. The protein lysate is derived from an established human carcinoma cell line (allogenic method). This lysate is incubated with the serum of a tumor patient or with pooled immunoglobulins of the G subclass (IgGs) of 100 non-tumor patients (commercially available from SIGMA company, Deisenhofen).
2. The protein lysate is derived from a tumor biopsy (autologous method). The lysate is incubated with serum of the same patient or with pooled sera of 100 non-tumor patients as under 1.

Common to both methods is that following incubation of the lysate with a serum an immunoprecipitation and a separation of the precipitated proteins in the 2D gel electrophoresis are carried out. Comparing the protein patterns resulting on the 2D gels of tumor patients and non-tumor patients in this way exclusively those proteins can be identified against which antibodies are present exclusively in sera derived from tumor patients. Thus, according to definition, every protein which is formed differentially and isolated via AMIDA is a tumor-associated antigen and is in principle suitable as a diagnostic marker for the detection of a tumor disease.

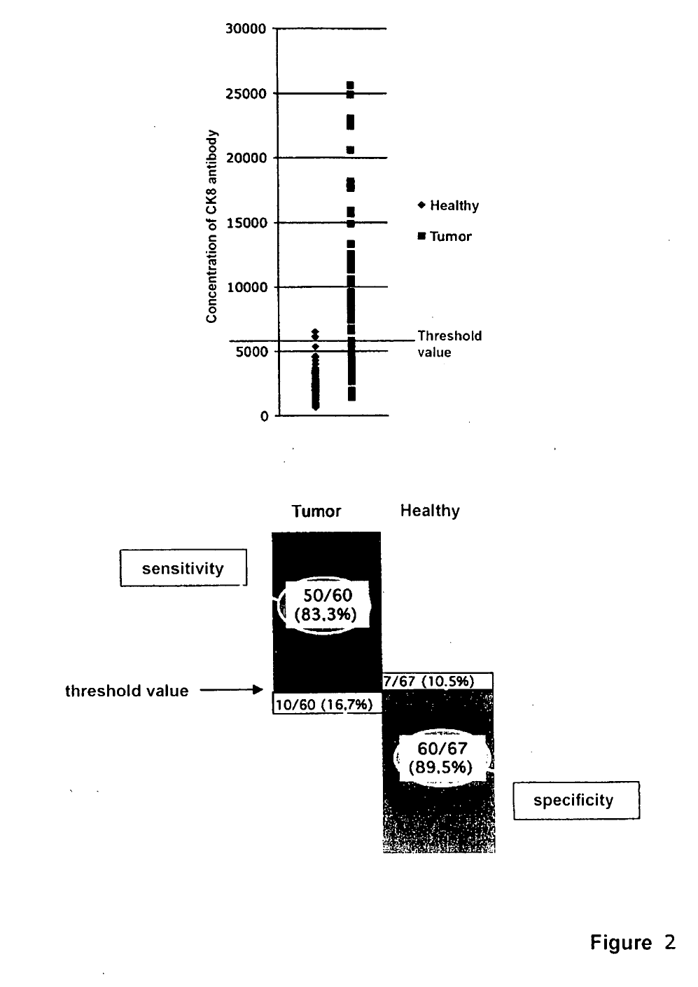

According to this principle, the inventors have carried out AMIDA with different tumor samples obtained from patients with tumors of the head-neck region, the intestine and the lung. One of the proteins isolated by the inventors is cytokeratin 8 (CK8) which has already been described as a tumor antigen. Similarly, CK8-specific antibodies have been described in patients with hepatocellular carcinomas, in oesophageal carcinomas and in hepatic autoimmune diseases. Since the inventors have isolated CK8 from the serum of a tumor patient they addressed the frequency of such antibodies in other tumor patients and also in non-tumor patients. For quantification of these antibodies the inventors used a method of detection wherein coloured beads were coated with recombinant CK8 protein. These beads were incubated with sera from tumor and non-tumor patients. The antibodies bound to the CK8 beads were detected with a fluorescein-coupled secondary antibody (mouse anti-human IgG) and quantified in a Bio-Plex measuring device. It is shown in FIG. 2 that tumor patients have significantly more CK8 antibodies in their serum than non-tumor patients.

In summary, the targeting of cells in vivo and in vitro using immunological tools such as T cells and antibodies, thus, is very much dependent on the knowledge of specific proteins on target cells.

The identification of molecular diagnostics for the early recognition of tumors has become a particular challenge due to the high mortality caused by cancer. Therefore, protein approaches have undergone a strong revival and their importance with respect to target-protein identification is well appreciated (HANASH, 2003). Targets, i.e. disease-specific markers, are useful for antibody-based approaches, DNA or protein vaccination and for the generation of antigen-specific cytotoxic T lymphocytes. In vivo the humoral immune reaction is one of the weapons of our immune system to fight against pathogenic and transformed cells.

Therefore, it is an object of the present invention to provide novel tumor antigens which can be employed as improved diagnostic markers in tumor recognition. It is another object of the invention to provide on the basis of these tumor antigens novel and improved therapies for the control of tumor diseases but in particular of the squamous epithelial carcinoma.

These objects have been achieved by the subject matter of the independent claims. Preferred embodiments can be seen from the dependent claims.

Once isolated and characterized the tumor antigens according to the invention can be used in many ways: among others (i) they can be used to load antigen presenting cells (APCs) such as for example dendritic cells or B cells and can thus be used for the activation of specific T cells. (ii) Monoclonal and bispecific antibodies can be prepared which are of high value for the diagnosis of a disease and the therapy of patients. (iii) The antigens can be employed as peptides or as DNA vaccines, respectively. Among others, the present invention is based on the AMIDA method as described above for the identification of antigens associated with diseases wherein a humoral immune response is elicited and thereby specific antibodies are formed. This method is based on precipitation mediated by autologous, allogenic or xenogenic antibodies of antigens from cell lysates or bacterial, parasitic and/or virus preparations with autologous, allogenic and/or xenogenic sera, ascites or pleural fluids. The induction of an immune response associated with the production of antibodies, thus, is the only prerequisite for the application of AMIDA. Therefore, the method is in particular suitable for the identification of tumor antigens but also for such antigens associated with autoimmune diseases or bacterial, viral and parasitic infections.

The term “antigens” as used in this specification refers to structures against which an organism forms antibodies because they are foreign to its immune system. Knowledge of antigens which are as specific as possible is am important prerequisite for diagnosis and immune therapy of tumor patients and individuals suffering e.g. from an autoimmune diseases or a chronic infection. Such antigens which are more or less specific for the disease in question enable the detection as well as targeting in vivo and in vitro of tumor cells, of cells being the target of an autoimmune reaction and also of infected cells and infectious organisms.

Therefore, the key features of the invention are the diagnostic detection of antigens and of autoantibodies directed against the same, respectively, by means of the tumor antigens according to the invention and the utilization of the antigens for therapy purposes.

invention provides a novel and promising tool for the therapy and diagnosis of a plurality of diseases and can also provide an important contribution to the development of novel therapies for these diseases.

Among the antigens identified by the inventors there are 27 proteins not known as tumor antigens up to now. These proteins are listed in Table 1.

TABLE 1
27 antigens were isolated via AMIDA from tumor biopsies isolated from
patients with a tumor disease of the head-neck region und from carcinoma cell lines.

| SEQ | | | | |
|---|---|---|---|---|
| ID NO: | Acc. N^{o} | protein | ID score | Reference |
| 1 | Q9p2V9 | Elongin A2 | 66 | (7) |
| 2 | BAA86587 | KIAA1273/TOB3 = KIAA1273/TOB3 | 100 | (14) |
| | | (AAH18701, NP 114127, AAH09938, | | |
| | | AAH02542) | | |
| 3 | Q13347 | TGF-beta receptor interacting protein 1 | 168 | (17) |
| 4 | P29354 | growth factor receptor-bound protein 2 | 122 | (16) |
| 55 | Q8WXF1 | paraspeckle protein 1 alpha isoform | 186 | (18) |
| 6 | P31943 | heterogenous nuclear ribonucleoprotein H | 107 | (19) |
| 7 | 1KAX | Heat shock cognate protein 70 | 99 | a-Ag (2) |
| 8 | P08670 | Vimentin | 298 | a-Ag (3) |
| 9 | O60588 | c-NAP1 | 61 | a-Ag (4) |
| 10 | O00109 | Mutant keratin 9 | 110 | (5) |
| 11 | D42825 | Kruppel-type zink finger protein ZNF-70 | 76 | (6) |
| 12 | Q9UJD9 | Similar to RPS2 | 69 | TREMBL |
| 13 | AAA69899 | AHNAK nucleoprotein | 61 | a-Ag (1) |
| 14 | P06753 | Tropomyosin alpha | 72 | (8) |
| 15 | 1MABB | ATP synthase beta chain | 129 | (9) |
| 16 | O63042 | Small ribosomal protein 4 | 68 | (10) |
| 17 | S55024 | Nebulin | 87 | (11) |
| 18 | Q96ES1 | Similar to serine proteinase | 107 | (12) |
| 19 | C1HURB | Complement subcomponent C1r | 106 | (13) |
| 20 | Q96PV1 | KIAA1937 | 99 | (14) |
| 21 | Q9Y2K3 | KIAA1000 | 63 | (14) |
| 22 | Q9ULK3 | KIAA1217 | 65 | (14) |
| 23 | Q96LL7 | CDNA FLJ25393 | 64 | TREMBL |
| 24 | Q96I66 | Unknown protein | 72 | TREMBL |
| 25 | Q9B5S71 | Hyp. 41.3 kD protein | 96 | TREMBL |
| 26 | P32320 | cytidine deaminase | 86 | (15) |
| 27 | P35080 | profilin II | 108 | (20) |

All antigens mentioned in Table 1 were identified from tumor biopsies with a technology developed for the first time by us (AMIDA; Gires et al (2004) Profile identification of disease-associated humoral antigens using AMIDA, a novel proteomics-based technology. Cell Mol Life Sci 61, 1198-1207; Rauch et al (2004) Allogenic antibody-mediated identification of head and neck cancer antigens. Biochem Biophys Res Commun 323, 156-162). AMIDA is a technology for the identification of tumor antigens based on the presence of specific antibodies in blood (and other body fluids) of tumor patients. Only such antigens are isolated against which antibodies are present in the blood. This also applies to all antigens listed herein.

All antigens mentioned herein have a property in common. This property is their antigenicity leading to the induction of antibodies. Thus, these antigens form a homologous group of proteins with respect to their function which is a common and characteristic property of these antigens. By means of this property the antigens were unequivocally defined by our AMIDA technology. They represent functional homologues in a classical sense. Even if the physiological function of these antigens is not homogeneous (and in part not even known) all of the antigens mentioned herein are so-called tumor antigens which are basically suitable for the diagnostics and/or for targeted immune therapy.

The complete sequence information with respect to SEQ ID NO: 1-27 can be found in the sequence overview at the end of the specification.

Among others, the following methods are used according to the invention:
Antibodies recognizing one or more of the antigens mentioned in Table 1 are detected with suitable methods. As a source of these autoantibodies serves whole blood or derivatives thereof such as serum or plasma of a human individual.
The antigens themselves are detected and quantified by suitable methods. Both embodiments serve preferably for the detection of a tumor disease in a human individual.
Combinations of the antigens listed in Table 1 and/or autoantibodies recognizing these antigens are used for the detection of a tumor disease.
The combination of one or more of the antigens mentioned in Table 1 and/or autoantibodies against these antigens are used with other tumor markers. These ‘other tumor markers’ can be markers already known (such as PSA, CEA, etc.) but also markers which will be discovered in the future and which are of a diagnostic value or achieve a diagnostic value only in combination with one or more of the antigens contained in Table 1 or with autoantibodies against these antigens.

According to a first aspect the present invention relates to an immunogenic squamous epithelial carcinoma antigen comprising at least one of the proteins of SEQ ID NO: 1-27 or variants thereof wherein the variant comprises one or more additions, insertions, substitutions and/or deletions as compared to the corresponding protein of SEQ ID NO: 1-27, and wherein the immunogenic activity of the variant is essentially equal to the activity of the corresponding unmodified protein of SEQ ID NO: 1-27.

The term “immunogenic activity”, as used herein, relates to the immunogenic function of the proteins according to the invention. As mentioned above, the tumor antigens according to the invention are overexpressed in different forms of the squamous epithelial carcinoma and therefore are important targets for immune-based anti-cancer therapies as well as for the detection and early recognition, respectively, of such diseases. Thus, the tumor antigens as disclosed herein above are considered to such an extent that they are capable of inducing an immune reaction in mammals, preferably humans, to thus serve as a therapeutic agent. The term immunogenic activity in particular relates to the immunogenic function of the proteins according to the invention to elicit immune reactions, in particular immune reactions mediated by cytotoxic T cells.

The amino acid sequences according the present invention similarly comprise all sequences differing from the sequences disclosed herein by amino acid insertions, deletions and substitutions.

Amino acid “substitutions” preferably are the result of an exchange of one amino acid by another amino acid having similar structural and/or chemical properties also called conservative amino acid substitutions. Amino acid substitutions can be performed on the basis of a similarity in polarity, charge, solubility, hydrophobicity, hydrophilicity and/or the amphipathic nature of the residues involved. For example, apolar (hydrophobic) amino acids include alanine, leucine, isoleucine, valine, proline, phenylalanine, tryptophane, and methionine; polar neutral amino acids include glycine, serine, threonine, cysteine, tyrosine, asparagine and glutamine; positively charged (basic) amino acids include arginine, lysine and histidine; and negatively charged (acidic) amino acids include aspartic acid and glutamic acid.

“Insertions” or “deletions” usually are in the range of 1-3 amino acids. The permitted variation can be experimentally determined by systematically performing insertions, deletions or substitutions of amino acids in a protein using recombinant DNA techniques and examining the resulting recombinant variants with respect to their immunological activity. This requires only routine experimentation from those skilled in the art.

The term “variant” also comprises peptide fragments of the tumor antigens according to the invention such as those which can be for example used for the pulsing of APCs. Such peptide fragments preferably have 5-50, particularly preferred 7-20 and most preferably 8-15 amino acid residues.

It is basically preferred in the present application that the variants of the proteins according to the invention show at least 50%, particularly preferred at least 70%, most preferably at least 80% sequence identity to the peptides according to SEQ ID NO: 1-27.

As a particularly preferred tumor antigen has been found the tumor antigen KIAA1273/TOB3 (SEQ ID NO: 2) of so far unknown function with an excellent suitability as a tumor marker. KIAA1273/TOB3 showed a very strong overexpression in head and neck carcinomas while in healthy mucous membranes KIAA1273/TOB3 was expressed only in cells of the basal lamina in very low concentration. Therefore, KIAA1273/TOB3 is a novel marker for head and neck carcinomas. According to a second aspect the present invention relates to an isolated nucleic acid encoding one or more of the proteins according to claim 1.

The term “nucleic acid sequence” relates to a heteropolymer of nucleotides or to the sequence of these nucleotides. The terms “nucleic acid” and “polynucleotide” are used interchangeably herein and refer to a heteropolymer of nucleotides.

The term “isolated” as used herein with respect to nucleic acids relates to a naturally occurring nucleic acid not directly adjacent to the two sequences by which it is flanked (one at the 5′ end and one at the 3′ end) in the naturally occurring genome of the organism from which it is obtained.

For example, an isolated nucleic acid can be, without limitation, a recombinant DNA molecule of any length with the proviso that the nucleic acid sequences usually flanking this recombinant DNA molecule in a naturally occurring genome have been removed or are missing. Therefore, an isolated nucleic acid includes, without limitation, a recombinant DNA existing as a separate molecule (for example a cDNA or a genomic DNA fragment generated by PCR or treatment with a restriction endonuclease) independent of other sequences, and also a recombinant DNA introduced into a vector, an autonomously replicating plasmid, a virus (for example a retrovirus, adenovirus or herpes virus) or into the genomic DNA of a prokaryote or eukaryote. In addition, an isolated nucleic acid can include a recombinant DNA molecule being part of a hybrid or a fusion nucleic acid sequence.

The term “isolated” similarly includes any non-naturally occurring nucleic acid because non-naturally occurring nucleic acid sequences cannot be found in nature and have no directly adjacent sequences in a naturally occurring genome. For example, a non-naturally occurring nucleic acid such as for example a nucleic acid prepared by genetic engineering is considered as an isolated nucleic acid. A nucleic acid generated by genetic engineering can be prepared by using conventional cloning techniques or chemical nucleic acid synthesis techniques. Isolated non-naturally occurring nucleic acids can be independent of other sequences or be introduced into a vector, an autonomously replicating plasmid, a virus (for example a retrovirus, adenovirus or herpes virus) or into the genomic DNA of a prokaryote or eukaryote. In addition, a non-naturally occurring nucleic acid can include a nucleic acid molecule being part of a hybrid or a fusion nucleic acid sequence.

The polynucleotides of the present invention also include, but are not limited to, a polynucleotide hybridising to the complement of the nucleotide sequences disclosed under moderately stringent or stringent conditions; a polynucleotide being an allelic variant of any of the above-described polynucleotides; a polynucleotide encoding a species homolog of any of the proteins disclosed herein; or a polynucleotide encoding a polypeptide carrying an additional specific domain or a truncation of the proteins disclosed.

The stringency of the hybridisation, as used herein, relates to conditions under which polynucleotide double strands are stable. As known to those skilled in the art, the stability of a double strand is a function of the concentration of sodium ions and of the temperature (see for example Sambrook et al., Molecular Cloning: A Laboratory Manual 2^{nd} Ed. (Cold Spring Harbor Laboratory, (1989)). The stringency levels used for hybridisation can be slightly modified by those skilled in the art.

The term weakly stringent hybridisation refers to conditions equivalent to a hybridisation in 10% formamide, 5× Denhart's solution, 6×SSPE, 0.2% SDS at 42° C., followed by washing in 1×SSPE, 0.2% SDS at 50° C. Denhart's solution and SSPE are well known to those skilled in the art as are other suitable hybridisation buffers.

A moderately stringent hybridisation relates to conditions enabling the binding of DNA to a complementary nucleic acid which has about 60% identity, preferably about 75% identity, particularly preferred about 85% identity to this DNA; wherein an identity of more than about 90% to this DNA is particularly preferred. Moderately stringent conditions are preferably conditions corresponding to a hybridisation in 50% formamide, 5× Denhart's solution, 5×SSPE, 0.2% SDS at 42° C. followed by washing in 0.2×SSPE, 0.2% SDS at 65° C.

Highly stringent hybridisation means conditions enabling only the hybridisation of those nucleic acid sequences which form stable double strands in 0.018 M NaCl at 65° C. (i.e. if a double strand is not stable in 0.018 M NaCl at 65° C. it is not stable under den highly stringent conditions as considered herein).

Furthermore, nucleic acid hybridisation techniques can be used to identify and obtain a nucleic acid in the scope of the present invention. Shortly, any nucleic acid having a certain homology to one of the sequences described in the present invention or a fragment thereof can be used as a probe for the identification of a similar nucleic acid by hybridisation under moderately stringent to highly stringent conditions. Such similar nucleic acids can then be isolated, sequenced and analysed to determine whether they are within the scope of the invention as described herein.

The invention also relates to an immunogenic antigen encoded by a nucleic acid as defined above.

According to a third aspect the invention comprises vectors comprising one or more of the nucleic acids as defined above.

Preferably, this is an expression vector comprising one or more of the nucleic acid sequences as defined herein above and one or more regulatory sequences. This expression vector preferably comprises one or more regulatory sequences. The term “expression vector” in general relates to a plasmid or a phage or a virus or a vector for expressing a polypeptide from a DNA (RNA) sequence. An expression vector can comprise a transcriptional unit containing the sequence of the following: (1) a genetic element or elements with a regulatory role in gene expression, for example promoters and/or enhancers, (2) a structural sequence or coding sequence which is transcribed into mRNA and translated into a protein; and (3) suitable transcriptional start and termination sequences. Structural units designed for the use in yeasts or higher eukaryotic expression systems preferably include a leader sequence enabling the extracellular secretion of a translated protein by a host. Alternatively, if expressed without a leader or transport sequence, a recombinant protein can include an N-terminal methionine residue. This residue can or cannot be subsequently cleaved off from the expressed recombinant protein to provide the final product.

Furthermore, the present invention provides for hosts, for example host cells transformed to contain the polynucleotides according to the invention. The term “transformation” means the introduction of DNA into a suitable host cell so that the DNA is capable of being replicated either as an extrachromosomal element or by chromosomal integration depending on the expression vector used.

Such host cells can for example contain nucleic acids according to the invention introduced into the host cell using known methods for transformation in a stable or transient manner. The present invention furthermore provides host cells altered by means of genetic engineering to express the polynucleotides of the present invention wherein such polynucleotides are operably linked to a regulatory sequence heterologous for the host cell and directing the expression of the polynucleotides in the cell.

The host cell can be a higher eukaryotic host cell, such as for example a plant cell, a mammalian cell or a lower eukaryotic host cell, such as for example a yeast cell or can be an insect cell, or the host cell can be a prokaryotic cell, for example a bacterium. The introduction of the recombinant construct into the host cell can be achieved by potassium phosphate transfection, DEAE, dextrane-mediated transfection, lipofection or electroporation (Davis, L. et al., Basic Methods in Molecular Biology (1986)).

Most preferable are such cells which usually do not express the specific protein or which express the protein on a low endogenous level or in a low endogenous concentration, respectively. The proteins according to the invention can be expressed in mammalian cells, yeasts, bacteria or other cells under the control of suitable promoters.

Suitable cloning and expression vectors for the use with prokaryotic and eukaryotic hosts and the respective methods are described in Sambrook et al. in Molecular Cloning: A Laboratory Manual, 2^{nd} Edition, Cold Spring Harbor, N.Y. (1989).

The mammalian cell is preferably a CHO, COS, HeLa, 293T, HEH or BHK cell.

Bacterial cells comprise Streptococci, Staphylococci, E. coli, Streptomyces and Bacillus subtilis cells. E. coli and Bacillus subtilis are preferred. Fungal cells can also be used, such as for example yeast cells (from Saccharomyces, Schizosaccharomyces, Pichia) and Aspergillus cells. As the insect cells Drosophila S2 and Spodoptera frugiperda Sf9 or SF21 cells are preferred. As the plant cells there can be used cells from N. tabacum or Arabidopsis thaliana.

According to a fourth aspect the present invention provides antibodies or aptamers directed against one or more epitopes of a protein according to claim 1.

The term “antibodies”, as used herein relates to intact antibodies and also antibody fragments retaining a certain capability to selectively bind to an epitope. Such fragments include, without limitation, Fab, F(ab′)₂, recombinant “single chain” antibodies and Fv antibody fragments. The term “epitope” relates to any antigenic determinant on an antigen, binding to the paratope of an antibody. Epitope determinants usually consist of chemically active surface groups of molecules (for example amino acid or sugar residues) and generally show three-dimensional structural characteristics and also specific charge characteristics.

The antibodies according to the invention can be prepared using any known method. For example, the pure protein according to the invention or a fragment thereof can be provided and be used as an immunogen to elicit in an animal an immune reaction of the type that specific antibodies are generated.

The preparation of polyclonal antibodies is well known to those skilled in the art. See for example Green et al., Production of Polyclonal Antisera, in Immunochemical Protocols (Manson, editor), pages 1-5 (Humana Press 1992) and Coligan et al., Production of Polyclonal Antisera in Rabbits, Rats, Mice and Hamsters, in Current Protocols In Immunology, section 2.4.1 (1992). In addition, various immunological techniques for the purification and concentration of polyclonal antibodies as well as monoclonal antibodies are known to those skilled in the art (Coligan et al., Unit 9, Current Protocols in Immunology, Wiley Interscience, 1994).

The preparation of monoclonal antibodies is similarly well known to those skilled in the art. See for example Köhler & Milstein, Nature 256: 495 (1975); Coligan et al., sections 2.5.1-2.6.7; and Harlow et al., Antibodies. A Laboratory Manual, page 726 (Cold Spring Harbor Pub. 1988). Shortly, monoclonal antibodies can be obtained by injecting mice with a composition including the protein according to the invention whereafter the presence of antibody production is verified by examination of a serum sample, the spleen is removed to obtain B lymphocytes and the B lymphocytes are fused with myeloma cells for the generation of hybridomas, the hybridomas are cloned, the positive clones producing a monoclonal antibody to the protein are selected, and the antibodies are isolated from the hybridoma cultures. Monoclonal antibodies can be isolated and purified from hybridoma cultures by a plurality of well established techniques. Such isolation techniques include an affinity chromatography with protein A or G Sepharose, size exclusion chromatography and ion exchange chromatography. See for example Coligan et al., sections 2.7.1-2.7.12 and section “immunoglobuline G (IgG)”, in Methods In Molecular Biology, volume 10, pages 79-104 (Humana Press 1992).

The term “antibodies” in the sense of the present invention means that the respective antibody due to its antigen specificity elicits or supports a desired stimulation of the immune system of the patient which is desired in the context of the treatment of the respective disease.

In particular, immunostimulating antibodies in the sense of the present invention are such antibodies which cause a T cell activation. Particularly advantageous in this respect is an activation of cytotoxic T lymphocytes (so-called T killer cells).

The terms “antibody” and “antibodies” in the sense of the present invention comprises both polyclonal antibodies and also monoclonal antibodies, chimeric antibodies (single chain antibodies), humanised antibodies all of which can be present in a bound or soluble form as well as fragments of the antibodies mentioned above. Besides the fragments of antibodies according to the invention alone antibodies according to the invention can also be present in a recombinant form as fusion proteins with other (protein) components. Fragments as such or fragments of antibodies according to the invention as components of fusion proteins are typically prepared by the methods of enzymatic cleavage, protein synthesis or recombination methods well known to those skilled in the art. Antibodies according to the present invention, thus, refers to polyclonal, monoclonal, human or humanised or recombinant antibodies or fragments thereof, single chain antibodies or also synthetic antibodies.

The polyclonal antibodies are heterogeneous mixtures of antibody molecules prepared from sera of animals immunized with an antigen. The object of the invention, however, also includes polyclonal monospecific antibodies obtained after purification of the antibodies (for example over a column loaded with peptides from a specific epitope). A monoclonal antibody contains an essentially homogeneous population of antibodies specifically directed to antigens wherein the antibodies essentially have the same epitope binding sites. Monoclonal antibodies can be obtained—as mentioned above—by the methods known in the art (e.g. Köhler and Milstein, Nature, 256, 495-397, (1975); U.S. Pat. No. 4,376,110; Harlow and Lane, Antibodies: A Laboratory Manual Cold Spring, Harbor Laboratory (1988); Ausubel et al., (eds.), 1998, Current Protocols in Molecular Biology, John Wiley & Sons, New York). The description contained in the above-mentioned references is incorporated by reference into the disclosure of the present invention.

Furthermore, genetically engineered antibodies according to the invention can be prepared according to methods as described in the references mentioned above. Shortly, for this purpose antibody-producing cells are cultured and the mRNA is isolated at a sufficient optical density of the cells via cell lysis by means of guanidinium thiocyanate, acidification with sodium acetate, extraction with phenol, chloroform/isoamyl alcohol, precipitations with isopropanol and washing with ethanol in a known manner. Subsequently, cDNA is synthesized from the mRNA using reverse transcriptase. The cDNA synthesized can be inserted directly or after genetic manipulation, for example by “site directed mutagenesis”, introduction of insertions, inversions, deletions or base substitutions into suitable animal, fungal, bacterial or viral vectors and can be expressed in the respective host organisms. Bacterial or yeast vectors, such as pBR322, pUC18/19, pACYCIS4, lambda or yeast mu vectors are preferred for the cloning of the genes and the expression in bacteria such as E. coli or in yeast such as Saccharomyces cerevisiae, respectively.

Antibodies according to the invention can belong to one of the following immunoglobulin classes: IgG, IgM IgE, IgA, IgD and optionally a subclass of the classes mentioned above such as IgG subclasses or mixtures thereof. Preferred are IgG and its subclasses, such as for example IgG1, IgG2, IgG2a, IgG2b, IgG3 or IgGM. Particularly preferred are the IgG subtypes IgG1/k or IgG2b/k. A hybridoma cell clone producing monoclonal antibodies according to the invention can be cultured in vitro, in situ or in vivo. The generation of high titers of monoclonal antibodies is preferably performed in vivo or in situ.

The chimeric antibodies according to the invention are molecules containing different components which are derived from different animal species (e.g. antibodies having variable region derived from a mouse monoclonal antibody and a constant region of a human immunoglobulin). Chimeric antibodies are preferably employed to reduce the immunogenicity during the application on the one hand and to increase the yields in production on the other hand, e.g. murine monoclonal antibodies give higher yields from hybridoma cell lines but also result in a higher immunogenicity in humans so that preferably chimeric human/murine or completely humanised antibodies, respectively, are employed. Even more preferably is a monoclonal antibody combining in itself the hypervariable complementarity defining regions (CDRs) of a murine monoclonal antibody with the other regions of a human antibody. An antibody of this type is referred to as a humanised antibody. Chimeric antibodies and methods for their preparation are known from the prior art (Cabilly et al., Proc. Natl. Sci. USA 81: 3273-3277 (1984); Morrison et al. Proc. Natl. Acad. Sci USA 81: 6851-6855 (1984); Boulianne et al. Nature 312: 643-646 (1984); Cabilly et al., EP-A-125023; Neuberger et al., Nature 314: 268-270 (1985); Taniguchi et al., EP-A-171496; Morrion et al., EP-A-173494; Neuberger et al., WO 86/01533; Kudo et al., EP-A-184187; Sahagan et al., J. Immunol. 137: 1066-1074 (1986); Robinson et al., WO 87/02671; Liu et al., Proc. Natl. Acad. Sci USA, 84: 3439-3443 (1987); Sun et al., Proc. Natl. Acad. Sci UA 84: 214218 (1987); Better et al., Science 240: 1041-1043 (1988) and Harlow and Lane, Antibodies: A Laboratory Manual, supra. These references are also incorporated herein by reference.

The designation “antibodies” shall include both intact molecules and also fragments of the same. As fragments can be mentioned all truncated or altered antibody fragments with one or two antigen-complementary binding sites, such as antibody portions with a binding site formed from heavy and light chains corresponding to the antibody, such as Fv, Fab or F(ab′)2 fragments or single chain fragments (see above). Preferred are truncated double chain fragments, such as Fv, Fab or F(ab′)₂, Fab and F(ab′)₂, fragments lack an Fc fragment as present in a intact antibody and can therefore be transported faster in the blood circulation and show a relatively lower unspecific tissue binding than intact antibodies. Such fragments are usually prepared by proteolytic cleavage using enzymes such as e.g. papain (for the preparation of Fab fragments) or pepsin (for the preparation of F(ab′)₂ fragments) or are obtained by chemical oxidation or by genetic engineering of the antibody genes.

Such antibodies can be for example used for the treatment of cancer cells or cells inflicted by a pathogen by directing them against a surface determinant of a tumor cell or a pathogen and by coupling, according to a preferred embodiment, a toxin, for example ricin or Pseudonmonas toxins, to the antibody; an overview is e.g. presented in Valera (1994) Blood 83: 309 bis 317; Vitetta et al. (1993) Immunol. Today 14: 252 bis 259. Immunostimulating antibodies in the sense of the present invention are preferably prepared by recombinant DNA techniques. Particularly preferred immunostimulatory antibodies are multispecific, in particular bispecific, and/or multifunctional, in particular trifunctional. Particularly in the case of bispecific antibodies there can be mentioned recombinant antibody molecules prepared by recombinant techniques, for example scFv molecules (so-called “single chain antibodies”), diabodies etc. The principal structure of bispecific antibodies and immunoconjugates is for example explained in van Spriel et al. (2000) Immunol. Today 21: 391-397. It should be understood that bispecific antibodies can also be prepared by known hybridoma techniques. Methods for the preparation of multivalent and of bispecific antibody fragments are known to those skilled in the art and are e.g. described in Tomlinson and Holliger (2000) Meth. Enzymol. 326: pp. 461. A particularly preferred example of a bispecific antibody is a trifunctional bispecific antibody to the Fc portion of which, i.e. the portion of the antibody which is not directly involved in antigen binding, accessory immune cells can bind (see Zeidler et al.: British Journal of Cancer 2000, Journal of Immunology 1999).

There are no limitations with regard to the mode of administration of the antibody according to the invention. Therefore, the antibody can be administered intraperitoneally, systemically (intravenously or intraarterially, intramuscularly, intradermally, subcutaneously, intratumorally) but also selectively into or via, respectively, a defined organ. As an example of selective application into an or via an organ can be mentioned administration via the bone marrow (as an immunological organ) or via a superselective catheter into a vessel (artery) supplying the respective organ or by direct intratumoral application, respectively. As a specific example of application by means of a catheter there can be mentioned that into the A. hepatica for selective application into the liver or for systemic application after passing that organ, respectively. Other examples of organ-specific applications are those into the liver via the portal vein, into the kidney via the renal artery, intrathecal application in the case of cerebral tumors, into the colon area via mesenterial blood vessels, into the pancreas via the Truncus coeliacus and the A. mesenteria superior and in tumors on limbs via the respective arteries. Furthermore, also direct application into a tumor can be performed.

Accordingly, a pharmaceutical composition is also provided according to the invention comprising at least an antibody as defined above according to the above definition. The pharmaceutical composition of the present invention is in particular suitable for the treatment of the above-mentioned diseases. Optionally, the pharmacologically active components of the pharmaceutical composition according to the invention are present in combination with one or more carriers and/or auxiliary agents as explained in more detail below.

In the pharmaceutical composition according to the invention or in the use the according to the invention, respectively, the antibody is advantageously provided in suitable formulations. Such formulations are known to those skilled in the art and contain besides the therapeutically or immunostimulatory active substances, respectively, one or more pharmaceutically acceptable carriers and/or pharmaceutically acceptable vehicles. Respective ways for suitable formulation and preparation of such formulations are for example disclosed in “Remington's Pharmaceutical Sciences” (Mack Pub. Co., Easton, Pa., 1980) which is incorporated into the disclosure of the present invention in its entirety. For parenteral administration there are considered as carrier substances for example sterile water, sterile saline, polyalkylene glycols, hydrogenated naphthalenes and in particular biocompatible lactide polymers, lactide/glycolide copolymers or polyoxyethylene/polyoxypropylene copolymers. Pharmaceutical compositions according to the invention can contain filler substances or substances such as lactose, mannitol, substances for covalent linkage of polymers such as e.g. polyethylene glycol to immunostimulatory antibodies according to the invention, complexation with metal ions or inclusion of materials in or on particular preparations of polymeric compounds such as e.g. polylactate, polyglycolic acid, hydrogel or on liposomes, microemulsions, micelles, unilamellar or multilamellar vesicles, erythrocyte fragments or spheroblasts. The respective embodiments of the pharmaceutical compositions are chosen dependent on the physical behaviour, for example with respect to the solubility, stability, bioavailability or degradation. Controlled or constant release of the active components according to the invention includes formulation on the basis of lipophilic deposits (e.g. fatty acids, waxes, or oils). In the frame of the present invention there are also disclosed coatings of pharmaceutical compositions according to the invention or medicaments, respectively, containing the therapeutically active substances, namely coatings with polymers (e.g. polyoxamers or polyoxamines). Furthermore, therapeutically active substances or compositions according to the invention can have protective coatings, e.g. protease inhibitors or permeability enhancers. Preferred carriers usually are aqueous carrier materials wherein water for injection (WFI) or water buffered with phosphate, citrate, HEPES or acetate etc. is used and the pH is generally adjusted to 5.0 to 8.0 (preferably 6.5 to 7.5). The carrier or vehicle, respectively, will preferably in addition contain salt components, e.g. sodium chloride, potassium chloride or other components which for example render the solution isotonic. Furthermore, besides the components mentioned above the carrier or vehicle, respectively, can contain additional components such as humane serum albumine (HSA), polysorbate 80, sugar or amino acids etc.

The mode of administration and the dosage of the medicament or the pharmaceutical compositions, respectively, according to the invention are dependent on the nature of the disease to be controlled, optionally from the stage thereof, the antigen to be targeted and also on the body weight, age and the sex of the patient.

The concentration of the active components in the formulations according to the invention can be varied within a broad range. Doses according to the invention of the antibody vary in the range of about 1 μg to about 10 mg.

As an alternative to the antibody mentioned above, the pharmaceutical composition of the present invention can comprise one or more of the proteins according to the invention, one or more of the nucleic acids according to the invention, a vector as defined above, an APC or a T cell as defined below and a pharmaceutically acceptable carrier.

According to a fifth aspect the present invention relates to a hybridoma producing a monoclonal antibody having a binding specificity for a protein according to claim 1.

According to a sixth aspect the invention comprises an ex vivo method for the generation of a population of autologous or allogenic antigen presenting cells (APCs) capable of inducing an effective immune reaction against a protein according to the invention comprising the following steps:

a) Providing autologous or allogenic APCs;

b) Contacting the APCs with an effective amount of a peptide fragment of a protein according to the invention under conditions enabling the endocytosis, processing and presentation of the peptide fragments by these APCs; and

c) Isolating the APCs presenting the corresponding peptides.

Alternatively, an ex vivo method for the preparation of genetically engineered APCs capable of inducing an effective immune reaction against a protein according to the invention comprises the following steps:

a) Providing a nucleic acid encoding a protein according to the invention or a peptide fragment thereof,

b) Transfecting the APCs with the nucleic acid; and

c) Selecting APCs presenting the peptide fragments.

According to a preferable embodiment the nucleic acid in step a) is provided in a expression vector.

According to a seventh aspect the present invention relates to an antigen presenting cell (APC) obtainable according to any of the methods mentioned above.

According to one embodiment this APC is a dendritic cell or a B cell.

An eighth aspect of the invention relates to an ex vivo method for the detection and for the recovery of T cells specific for a protein according to the invention comprising the following steps:

a) Providing mammalian, in particular human, T cells or PBMCs;

b) Co-culturing the cells with an APC according to the seventh aspect of the present invention under conditions enabling an activation of T cells; and

c) Determining the presence of a specific activity of the T cells such APC; and

d) optionally selection and cultivation/expansion of such T cells which in step c) have shown a specificity for such APC as defined above.

The term activation as used herein is defined as the stimulation of T cells. The co-culturing step performed in b) is necessary to activate and, thus, to select specific T cells. This can be for example carried out by direct application of the proteins of the present invention onto a sample, preferably a blood sample obtained from the patient. Thus, peripheral blood mononuclear cells (═PBMCs) which contain T cells and can be easily prepared from patient blood samples can provide APCs (for example B cells) which are pulsed by the peptides according to the invention.

Alternatively, the pulsed APCs can be prepared separately before they are used in step b), and this by simply pulsing APCs, preferably autologous APCs, with the proteins according to the invention according to methods known in the prior art and then co-culturing the sample with the APCs. These APCs are preferably selected to express the suitable MHC class I molecule.

The T cells whose activity can be determined by the method according to the invention are preferably cytotoxic T cells, particularly preferred CD8+ T cells.

The activity of the T cells in the above step c) can be determined by methods known pei se in the prior art. Further information can be for example found in Immunology, 5^{th} edition, 2001.

According to one embodiment this determination can be carried out by means of a ⁵¹Cr release assay. In particular, the T cell function can be determined by a ⁵¹Cr release assay using a T cell bioassay based on the killing of a target cell by a cytotoxic T cell. This assay is based on the incorporation of radioactively labelled sodium chromate, Na₂⁵¹CrO₄, by living cells which do not spontaneously release this sodium chromate. If these labelled cells are killed, the radioactive chromate is released and its presence in the supernatant of mixtures from target cells and cytotoxic T cells can be measured. An example of target cells are T2 cells pulsed with one or more of the proteins according to the invention.

According to a preferable embodiment the determination in step c) is performed by measuring the amount of cytokins in the T cells. For this purpose the following method can be used:

First the cytokins can be measured by intracellular cytokin staining.

The intracellular cytokin staining approach is based on the use of metabolic toxins inhibiting protein export from the cell. Then, the cytokin accumulates within the endoplasmatic reticulum and the vesicular network of the cell. If the cells are subsequently fixed and permeabilised by using mild surface-active agents antibodies can gain access to these intracellular compartments and are capable of detecting the cytokin.

Alternatively, the cytokins are measured extracellularly by means of an ELISPOT assay. The ELISPOT assay is a modification of an ELISA antigen capture assay. It is an excellent approach to measure the frequency of T cell reactions. Populations of T cells are stimulated with the antigen of interest and are allowed to sit on plastic plates coated with antibodies and directed against the cytokin to be examined. All cytokins secreted by the T cell are captured by the antibody on this plastic plate. After a defined amount of time the cells are removed and a second antibody against the cytokin is added to the plate so that a circle of bound cytokin appears surrounding the position of every activated T cell wherein every spot is counted and the knowledge of the number of T cells originally added to the plate enables an easy calculation of the frequency of T cells secreting the specific cytokin.

The method for the generation of specific T cell lines is comprehensively described in Moosmann et al., B cells immortalized by a mini-Epstein-Barr virus encoding a foreign antigen efficiently reactivate specific cytotoxic T cells, Blood, 1. September 2002, Vol. 100, Nr. 5.

According to a ninth aspect the invention relates to T cells obtainable by the method as explained above.

According to a tenth the present invention relates to a diagnostic composition comprising one or more of the proteins according to the invention, an antibody as defined above or a T cell according to the invention.

This composition can be for example provided in the form of a test kit by which e.g. via the antibody according to the invention the corresponding tumor antigens are detected in a sample (e.g. biopsy) or by which via the tumor antigens according to the invention the autoantibodies of a patient to be examined are detected, or the T cells directed against an antigen according to the invention are determined e.g. by means of specific tetramers.

According to an eleventh aspect the invention relates to the use of the above-mentioned compositions in the diagnosis or therapy of a squamous epithelial carcinoma, in particular a squamous epithelial carcinoma in the otolaryngologic, head, and neck region, e.g. of the head-neck region.

Unless specified otherwise, all technical and scientific terms used herein have the meaning which is usually assigned to them by those skilled in the art in this field. All publications, patent applications, patents and other references mentioned herein are incorporated herein by reference in their entirety. In case of a conflict the present specification including the definitions is significant. Additionally, the Materials and Methods and the Examples are only illustrative and should not be construed as limiting.

In the following, the invention will be illustrated by means of Examples and referring to the accompanying Figures which show:

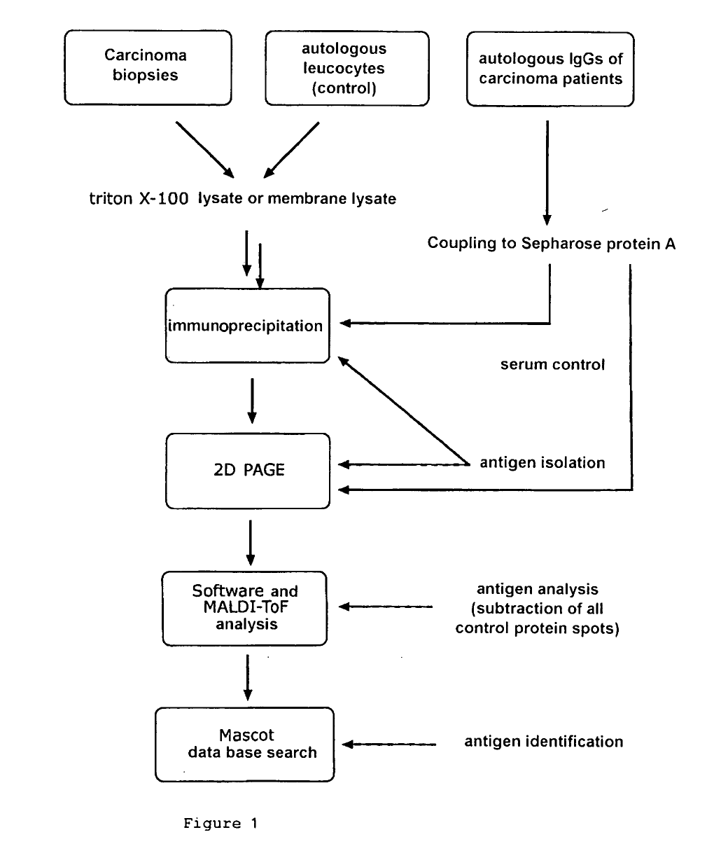

FIG. 1: The principle of the autologous version of AMIDA. By the immunoprecipitation step exclusively those proteins are isolated against which autoantibodies are present in sera of tumor patients and non-tumor patients. Autoantibodies which are only present in sera of patients can be used for the detection of a tumor disease. In the allogenic version, the cell lysate is obtained from an established tumor cell line. Then, allogenic sera from tumor patients are compared with those of non-tumor patients.

FIG. 2: CK8-specific antibodies are significantly increased in the blood of most tumor patients (orange) versus non-tumor patients (green). Following quantification in a modified ELISA system (and calculated as ‘relative light units’) a sensitivity of 83.3% and a specificity of 89.5% is obtained for the test.

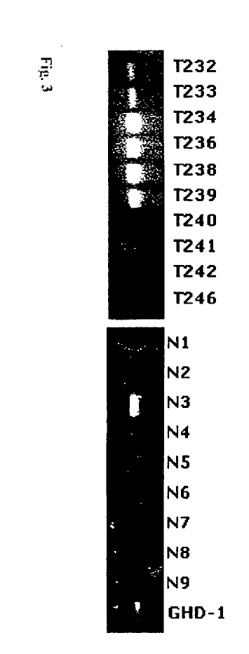

FIG. 3: KIAA1937 is expressed in all carcinomas of the head-neck region tested (T232-T246). In contrast, expression is not detectable in almost all healthy control tissues (N1, N2, N4-9). GHD-1 is the carcinoma cell line which served for the identification of KIAA1937 as a tumor antigen. It was derived from a hypopharynx carcinoma and was established in the laboratory of the inventors.

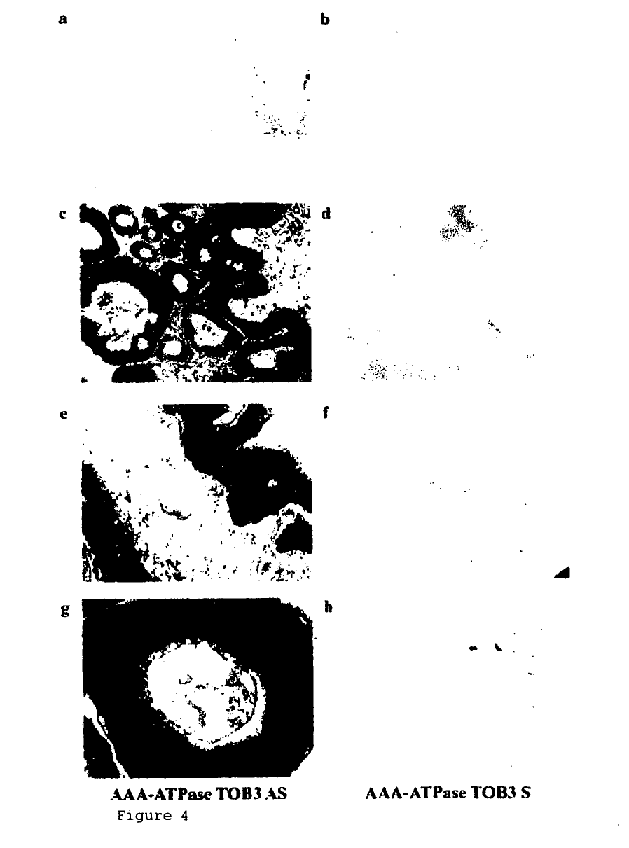

FIG. 4: KIAA1273/TOB3 mRNA expression in healthy mucous membrane and in carcinomas. Cryosections of healthy mucous membrane (a and b) and carcinoma samples (c-h) were prepared by in situ hybridisation using a labelled KIAA1273/TOB3/AAA ATPase KIAA1273/TOB3 complementary antisense RNA (AAA ATPase KIAA1273/TOB3 AS) and a labelled KIAA1273/TOB3/AAA ATPase KIAA1273/TOB3 sense RNA (AAA ATPase KIAA1273/TOB3 S) as a control. Healthy mucous membrane stained weakly with the complementary RNA in cells of the basal lamina whereas carcinoma cells showed a strong and specific staining.

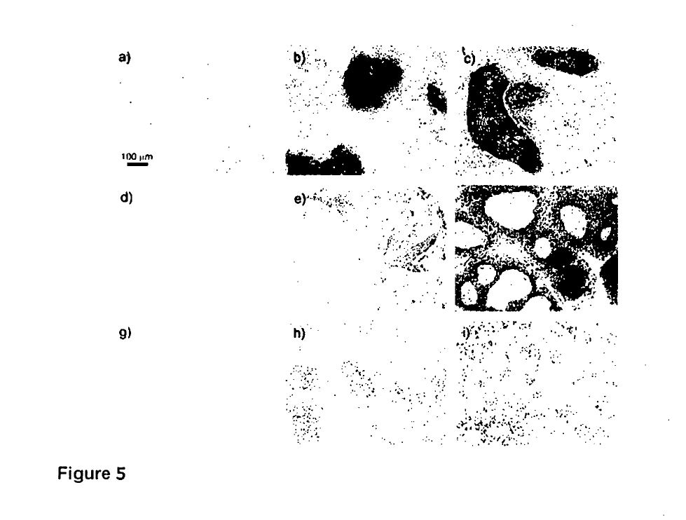

FIG. 5: Immunohistochemical detection of e-FABP (a-c), Grb2 (d-f) and hnRNP H (g-i) in healthy buccal mucous membrane (a, d, g), primary carcinomas of the head-neck region (b, e, h) and metastases derived therefrom (c, f, i).

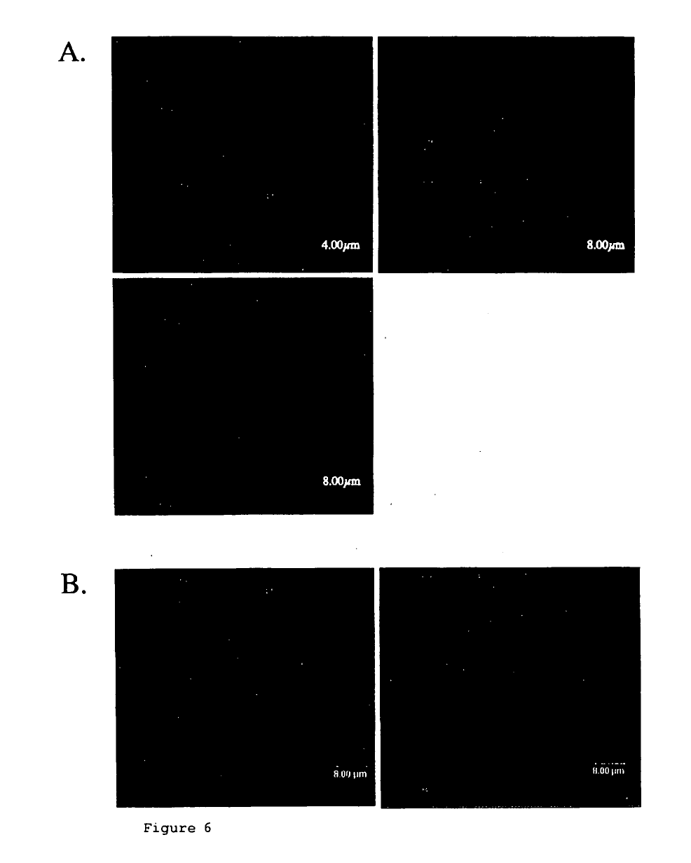

FIG. 6: Detection of CK8 expression at the cell surface. FaDu cells were treated with sucrose/EGS and in this manner the plasma membrane was impermeabilised. Subsequently, antibodies specific for CK8 or actin, respectively, were used in combination with FITC or Texas Red conjugated secondary antibodies, respectively, for the detection of both molecules. (B) FaDu cells were permeabilised with Triton X-100 and actin was detected with specific antibodies. Shown are confocal laser scanning micrographs of the staining.

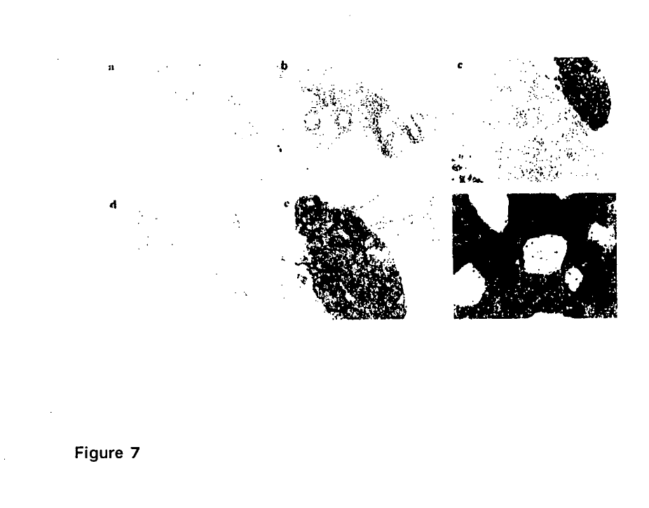

FIG. 7: CK8 detection in autologous tissues. Cryosections of healthy (a, d), hyperplasic (b) and tumor tissue (c, e) or metastases (f), respectively, of a patient were prepared and stained with CK8-specific antibodies. The expression of CK8 increased with increasing transformation of the tissue.

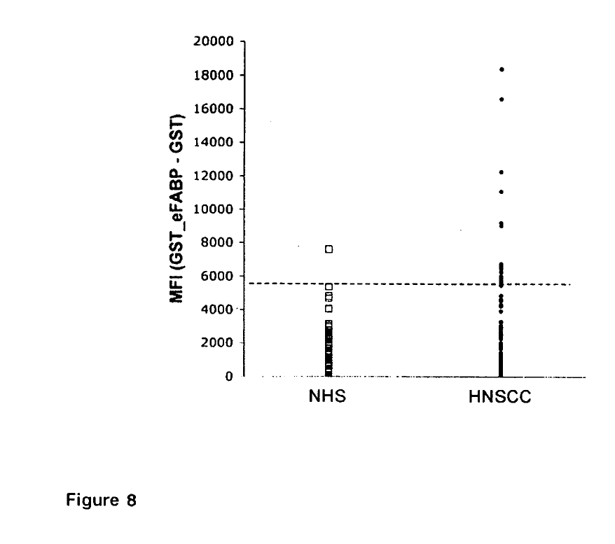

FIG. 8: The E-FABP serum reactivity was determined for normal human sera (NHS, n=48) and HNSCC patients (n=59) with recombinant E-FABP in a modified Bio-Plex approach. Shown are the mean values of the fluorescence from three independent experiments.

### EXAMPLES

1. Identification of Tumor-Associated Antigens using AMIDA

Sera of patients suffering from a squamous epithelial carcinoma of the upper airways and oesophagus were collected together with samples of the autologous tumors and subjected to an AMIDA screening procedure as schematically depicted in FIG. 1. Cancer cell biopsies were processed into single cell suspensions and a defined cell number was lysed to obtain raw protein preparations or membrane-associated protein fractions. Potential tumor-associated antigens (TAAs) were obtained by immunoprecipitations (IP) with immobilised autologous serum antibodies over night and separated by 2DE as described in the experimental procedures of the PCT application (WO 03/025568) mentioned above. As a control autologous leucocytes were processed as a protein reference in parallel with the tumor material. Serum antibodies coupled to Sepharose A beads were processed in the same manner and served as a second negative control. In addition, an AMIDA “screen” was performed in the allogenic manner. For this purpose two established otolaryngologic, head, and neck carcinoma cell lines served as a protein source. The proteins were immunoprecipitated with immobilised antibodies according to the allogenic AMIDA variant (see above) and separated in a 2DE. As a control, the proteins were also immunoprecipitated with pooled IgGs of 100 healthy donors. An analysis of the 2DE protein patterns and determination of different protein spots was performed using the Imagemaster 2DE software (FIGS. 1 and 2). Tumor IP-specific protein spots were excised, subjected to tryptic digestion in-gel and the resulting peptides were examined by means of MALDI-TOF mass spectrometry. A total of six squamous epithelial carcinomas of the upper airways and oesophagus were examined independently of each other in an autologous AMIDA procedure and the IgGs of 8 carcinoma patients tested in an allogenic AMIDA procedure against two tumor cell lines. By the combination of both variants of the AMIDA method it was possible to identify 27 potential tumor-associated antigens (see Table 1).

In situ hybridisation—Frozen tissue sections (5 μm) were fixed in 4% paraformaldehyde and washed in PBS, dehydrated in ethanol and stored at −70° C. After thawing, inactivation of endogenous alkaline phosphatase with HCl (0.2 N), and digestion with proteinase K (10 μg/mL), the microscopic slides were treated with 0.1 M glycine/0.05 M PBS and 4% paraformaldehyde at RT. Washing steps with PBS were performed between each treatment. Afterwards, sections were permeabilised with 0.1 M triethanolamine/0.25% acetic anhydride, washed with 2×SSC and incubated in pre-hybridisation buffer (5 h, RT, 50% deionized formamide, 4×SSC, 5× Denhardt's, 25 μg/ml salmon sperm DNA, 0.1% SDS, 50 μg/ml t-RNA, 5% dextrane sulfate). KIAA1273/TOB3 cDNA was amplified by PCR using the forward primer 5′-CGATGGTACCGATCCTGGGTGCAGATGCAGCTGGAAG-3′ and the reverse primer 5′-ATCGCTCGAGCTACAACAGGGGGTGCCCTGGGGG-3′ with RZPD clone IRALp962O1117 serving as a template. The PCR product was cloned into pDrive vector (Qiagen, Hilden, Germany). pDrive-KIAA1273/TOB3 was linearised by BamHI or alternatively HindIII digestion for an in vitro transcription to generate sense and antisense DIG-labelled RNA samples using the DIG RNA Labeling Kit (Roche, Mannheim, Germany) including T7 and SP6 RNA polymerases. The resulting samples were diluted in pre-hybridisation buffer for hybridisation of the sections over night at 70° C. After stringent washing (2×SSC and 0.2×SSC) the sections were further washed with T1 buffer (0.5 M maleic acid, 750 mM NaCl, pH 7.5) and incubated in T2 buffer (T1 buffer+1% blocking reagent, 1 h, RT). Subsequently, the sections were incubated (1 h, RT) with an alkaline phosphatase conjugated anti-DIG antibody (Roche, Mannheim, Germany). The signals were visualised using nitro blue tetrazolium (NBT) and 5-bromo-4-chloro-3-indolyl phosphate (BCIP) as a substrate (Roche, Mannheim, Germany). The reaction was stopped by washing the microscopic slides in PBS buffer. Control experiments were performed in parallel for each test using DIG-labelled sense RNA probes.

2. KIAA1273/TOB3 is Specifically Expressed in Carcinoma Cells

AMIDA screening enabled the isolation and identification of KIAA1273/TOB3 (KIAA1273/TOB3, SEQ ID NO: 2) and the verification of its tumor specificity. A weak KIAA1273/TOB3 mRNA expression was observed in the basal lamina layer of healthy mucous membrane whereas higher differentiated epithelia did no longer express KIAA1273/TOB3 mRNA (FIG. 4a). In sharp contrast, carcinoma cells of neck and head samples expressed high amounts of the mRNA for KIAA1273/TOB3 (FIG. 4c, e, g). Therefore, KIAA1273/TOB3 was expressed de novo in carcinoma cells of the upper airways and oesophagus. KIAA1273/TOB3 is a newly identified member of the TIS21/PC3/BTG1/-TOB family containing a putative AAA ATPase motif with unknown function (Parng, C. et al.) and regulated in its expression by the c-myc proto-oncogen. Presumably, KIAA1273/TOB3 overexpression results from a direct effect of the c-myc regulation in cancer cells since c-myc is also often overexpressed in tumors.

3. Immunohistochemical Detection of the Overexpression of Grb2, hnRNP H and E-FABP in Head-Neck Carcinomas

GRB2, hnRNP H and E-FABP were isolated as potential tumor-associated antigens by means of the AMIDA technology. Subsequently, the expression of these three antigens was examined in healthy tissue, in carcinomas of the head-neck region and in metastases derived therefrom. Ultra-thin sections of cryosamples of the corresponding tissues were prepared and subjected to immunohistochemical staining with specific antibodies. Grb2, hnRNP H and E-FABP were strongly overexpressed both in primary carcinomas and also in local regional metastases as compared to healthy tissue (FIG. 5). The overexpression of E-FABP and hnRNP H was examined in a number of healthy tissues and carcinomas. Both AMIDA antigens showed a clear overexpression in transformed tissue (Tables 2 and 3).

TABLE 2
Expression pattern of E-FABP in HNSCC.

| | Expression pattern | | | |
|---|---|---|---|---|
| Localisation | − | + | ++ | +++ |
| Pharynx | 0/11 (0%) | 4/11 (36%) | 2/11 (18%) | 5/11 (46%) |
| Larynx | 0/2 (0%) | 0/2 (0%) | 1/2 (50%) | 1/2 (50%) |
| Metastases | 0/5 (0%) | 0/5 (0%) | 1/5 (20%) | 4/5 (80%) |
| Healthy | 0/3 (0%) | 3/3 (100%) | 0/3 (0%) | 0/3 (0%) |
| mucous | | | | |
| membrane | | | | |

+++ strong expression;
++ moderate expression;
+ weak expression;
− no expression.

TABLE 3
Expression pattern of hnRNP H in HNSCC

| | Expression pattern | | | |
|---|---|---|---|---|
| Localisation | 0-25%* | 26-50%* | 51-75%* | 76-100%* |
| Oropharynx | 0/5 (0%) | 0/5 (0%) | 2/5 (40%) | 3/5 (60%) |
| Hypopharynx | 1/5 (20%) | 0/5 (0%) | 0/5 (0%) | 4/5 (80%) |
| Larynx | 0/3 (0%) | 0/3 (0%) | 0/3 (0%) | 3/3 (100%) |
| Metastases | 0/3 (0%) | 1/3 (33%) | 0/3 (0%) | 2/3 (67%) |
| Healthy | 8/8 (100%)** | 0/8 (0%) | 0/8 (0%) | 0/8 (0%) |
| mucous | | | | |
| membrane | | | | |

*Percentage of hnRNPH-positive cells in tissue samples.
**with the exception of the basal lamina layer in which hnRNPH was constitutively expressed.

4. Examination of CK8 Overexpression in Healthy and Transformed Epithelia

Cytokeratin 8 (CK8) is an intermediary filament protein causing a humoral response in vivo in HNSCC patients. In contrast to healthy epithelia CK8 is expressed in carcinomas also on the outside of the plasma membrane as shown by flow cytometry. Although several references describe such phenomenon, i.e. the presence of keratins on the cell surface (21-26), this is still a matter of controversial discussion (27). For this reason FaDu carcinoma cells were treated with a mixture of sucrose/EGS (ethyleneglycol bis-succinimidyl succinate), and in this way the plasma membrane was impermeabilised. Subsequently, CK8 and, as a control, actin were immunohistochemically detected and visualised in a confocal laser scanning microscope. As expected, actin could not be detected in treated cells although the antibody used was able to stain actin in permeabilised cells. In contrast, CK8 could be detected in sucrose/EGS treated cells on the outer face of the plasma membrane (FIG. 6).

Furthermore, the expression of in healthy, hyperplasic and tumor tissue of a patient was immunohistochemically examined. Cryosections were generated and stained with specific antibodies. CK8 was weakly detectable in cells of the basal lamina in healthy tissue. Already with beginning hyperplasia a clear overexpression of CK8 in the tissue occurred which increased in the course of tissue transformation (FIG. 7).

5. Determination of E-FABP-Specific Serum Reactivity in Healthy Individuals and Cancer Patients

E-FABP was identified in an allogenic AMIDA approach with serum antibodies from three out of eight patients. The serum reactivity against E-FABP was determined in a modified Bio-Plex approach for 48 healthy blood donors and 59 HNSCC patients. With a calculated specificity of 95% the E-FABP-specific serum reactivity reached a sensitivity of 22%, i.e 22% of the cancer patients were specifically diagnosed in a retrospective manner (FIG. 8).

```
Sequence overview of SEQ ID NO: 1-27 (Assignment see Table 1):
AHNAK nucleoprotein
      1 mpgikvggsg vnvnakgldl ggrggvqvpa vdissslggr pvevqgpsle sgdhakikfp
    61 tmkvpkfgvs tgregqtpka glrvsapevs vghkggkpgl tiqapqlevs vpsaniegle
  121 gklkgpqitg pslegdlglk gakpqghigv dasapqiggs itgpsvevqa pdidvqgpgs
  181 klnvpkmkvp kfsvsgakge etgidvtlpt gevtvpgvsg dvslpeiatg glegkmkgtk
  241 vktpemiiqk pkismqdvdl slgspklkgd ikvsapgvqg dvkgpqvalk gsrvdietpn
  301 legtltgprl gspsgktgtc rismsevdln vaapkvkggv dvtlprvegk vkvpevdvrg
  361 pkvdvsapdv eahgpewnlk mpkmkmptfs tpgakgegpd vhmtlpkgdi sisgpkvnve
  421 apdvnleglg gklkgpdvkl pdmsvktpki smpdvdlhvk gtkvkgeydv tvpklegelk
  481 gpkvdidapd vdvhgpdwhl kmpkmkmpkf svpgfkaegp evdvnlpkad vdisgpkidv
  541 tapdvsieep egklkgpkfk mpemnikvpk ismpdvdlhl kgpnvkgeyd vtmpkvesei
  601 kvpdvelksa kmdidvpdve vqgpdwhlkm pkmkmpkfsm pgfkaegpev dvnlpkadvd
  661 isgpkvgvev pdvniegpeg klkgpkfkmp emnikapkis mpdvdlhmkg pkvkgeydmt
  721 vpklegdlkg pkvdvsapdv emqgpdwnlk mpkikmpkfs mpslkgegpe fdvnlskanv
  781 disapkvdtn apdlslegpe gklkgpkfkm pemhfrapkm slpdvdldlk gpkmkgnvdi
  841 sapkiegemq vpdvdirgpk vdikapdveg qgldwslkip kmkmpkfsmp slkgegpevd
  901 vnlpkadvdv sgpkvdieap dvslegpegk lkgpkfkmpe mhfktpkism pdvdlhlkgp
  961 kvkgdvdvsv pkvegemkvp dveikgpkmd idapdvevqg pdwhlkmpkm kmpkfsmpgf
1021 kgegrevdvn lpkadidvsg pkvdvevpdv slegpegklk gpkfkmpemh fkapkismpd
1081 vdlnlkgpkl kgdvdvslpe vegemkvpdv dikgpkvdis apdvdvhgpd whlkmpkvkm
1141 pkfsmpgfkg egpevdvklp kadvdvsgpk mdaevpdvni egpdaklkgp kfkmpemsik
1201 pqkisipdvg lhlkgpkmkg dydvtvpkve geikapdvdi kgpkvdinap dvevhgpdwh
1261 lkmpkvkmpk fsmpgfkgeg pevdmnlpka dlgvsgpkvd idvpdvnlea pegklkgpkf
1321 kmpsmniqth kismpdvgln lkapklktdv dvslpkvegd lkgpeidvka pkmdvnvgdi
1381 diegpegklk gpkfkmpemh fkapkismpd vdlhlkgpkv kgdmdvsvpk vegemkvpdv
1441 dikgpkvdid apdvevhdpd whlkmpkmkm pkfsmpgfka egpevdvnlr kadidvsgps
1501 vdtdapdldi egpegklkgs kfkmpklnik apkvsmpdvd lnlkgpklkg eidasvpele
1561 gdlrgpqvdv kgplveaevp dvdlecpdak lkgpkfkmpe mhfkapkism pdvdlhlkgp
1621 kvkgdadvsv pklegdltgp svgvevpdve lecpdaklkg pkfkmpdmhf kapkismpdv
1681 dlh
Heat shock cognate protein 70
      1 mskgpavgid lgttyscvgv fqhgkveiia ndqgnrttps yvaftdterl igdaaknqva
    61 mnptntvfda mrligrrfdd avvqsdmkhw pfmvvndagr pkvqveykge tksfypeevs
  121 smvltkmkei aeaylgktvt navvtvpayf ndsqrqatkd agtiaglnvl riineptaaa
  181 iaygldkkvg aernvlifdl gggtfdvsil tiedgifevk stagdthlgg edfdnrmvnh
  241 fiaefkrkhk kdisenkrav rrlrtacera krtlssstqa sieidslyeg idfytsitra
  301 rfeelnadlf rgtldpveka lrdakldksq ihdivlvggs tripkiqkll qdffngkeln
  361 ksinpdeava ygaavqaail s
Vimentin
      1 mstrsvssss yrrmfggpgt asrpsssrsy vttstrtysl gsalrpstsr slyasspggv
    61 yatrssavrl rssvpgvrll qdsvdfslad aintefkntr tnekvelqel ndrfanyidk
  121 vrfleqqnki llaeleqlkg qgksrlgdly eeemrelrrq vdqltndkar veverdnlae
  181 dimrlreklq eemlqreeae ntlqsfrqdv dnaslarldl erkveslqee iaflkklhee
  241 eiqelqaqiq eqhvqidvdv skpdltaalr dvrqqyesva aknlqeaeew ykskfadlse
  301 aanrnndalr qakqesteyr rqvqsltcev dalkgtnesl erqmremeen faveaanyqd
  361 tigrlqdeiq nmkeemarhl reyqdllnvk maldieiaty rkllegeesr islplpnfss
  421 lnlretnids lplvdthskr tfliktvetr dgqvinetsq hhddle
c-NAP1
      1 metrspglnn mkpqslqlvl eeqvlalqqq maenqaaswr klknsqeaqq rqatlvrklq
    61 akvlqyrswc qelekrleat ggpipqrwen veepnldell vrleeeqqrc eslaevntql
  121 rlhmekadvv nkalredvek ltvdwsrard elmrkesqwq meqeffkgyl kgehgrllsl
  181 wrevvtfrrh flemksatdr dlmelkaehv rlsgslltcc lrltvgaqsr epngsgrmdg
  241 repaqilill aktqelekea hersqeliql ksqgdlekae lqdrvtelsa lltqsqkqne
  301 dyekmikalr etveiletnh telmeheasi srnaqeekls lqqvikditq vmveegdnia
  361 qgsghensle ldssifsqfd yqdadkaltl vrsvltrrrq avqdlrqqla gcqeavnllq
  421 qqhdqweeeg kalrqrlqkl tgerdtlagq tvdlqgevds lskerellqk areelrqqle
  481 vleqeawrlr rvnvelqlqg dsaqgqkeeq qeelhlavre rerlqemlmg leakqsesls
  541 elitireale sshlegellr qeqtevtaal araeqsiael sssentlkte vadlraaavk
  601 isalnealal dkvglnqqll qleeenqsvc srmeaaeqar nalqvdlaea ekrrealwek
  661 nthleaqlqk aeeagaelqa dirdiqeeke eiqkklsesr hqqeaattql eqlhqeakrq
  721 eevlaravqe kealvrekaa levrlqaver drqdlaeqlq gissakelle sslfeaqqqn
  781 svievtkgql evqiqtvtqa keviqgevrc lkleldters qaeqerdaaa rqlaqaeqeg
  841 ktaleqqkaa hekevnqlre kwekerswhq qelakalesi erekmelemr lkeqqtemea
  901 iqaqreeert qaesalcqmq letekervsl letllqtqke ladasqqler lrqdmkvqkl
  961 keqettgilq tqlqeaqrel keaarqhrdd laalqeesss llqdkmdlqk qvedlksqlv
1021 aqddsqrlve qevqeklret qeynriqkel erekasitis lmekeqrllv lqeadsirqq
1081 elsalrqdmq eaqgeqkels aqmellrqev kekeadflaq eaqlleelea shiteqqlra
1141 slwaqeakaa qiqirirste sqlealaaeq qpgnqaqaqa qlaslysalq qalgsvcesr
1201 pelsgggdsa psvwglepdq ngarslfkrg plltalsaea vasalhklhq dlwktqqtrd
1261 vlrdqvqkle erltdteaek sqvhtelqdl qrqlsqnqee kskwegkqns leselmeihe
1321 tmaslqsrlr raelqrmeaq gerellqaak enltaqvehl qaavvearaq asaagileed
1381 irtarsalki kneevesere raqalqeqge lkvaqgkalq enlalltqtl aereeevetl
1441 rgqiqelekq remqkaalel lsldlkkrnq evdlqqeqiq elekcrsvle hlpmavqere
1501 qkltvqreqi relekdretq rnvlehqlle lekkdqmies qrgqvqdlkk qlvtleclal
1561 eleenhhkme cqqklikele gqretqrval thitidleer sqelqaqssq ihdleshstv
1621 larelqerdq evksqreqie elqrqkehlt qdlerrdqel mlqkeriqvl edqrtrqtki
1681 leedleqikl slrergrelt tqrqlmqera eegkgpskaq rgslehmkli lrdkekevec
1741 qqehihelqe lkdqleqqlq glhrkvgets lllsqreqei vvlqqqlqea reqgelkeqs
1801 lqsqldeaqr alaqrdqele alqqeqqqaq gqeervkeka dalqgaleqa hmtlkerhge
1861 lqdhkeqarr leeelavegr rvqaleevlg dlraesreqe kallalqqqc aeqaqeheve
1921 tralqdswlq aqavlkerdq elealraesq ssrhqeeaar araealqeal gkahaalqgk
1981 eqhlleqael srsleastat lqasldacqa hsrqleealr iqegeiqdqd lryqedvqql
2041 qqalaqrdee lrhqqereql lekslaqrvq enmiqekqnl gqereeeeir glhqsvrelq
2101 ltlaqkeqei lelretqqrn nlealphshk tspmeeqslk ldsleprlqr elerlqaalr
2161 qteareiewr ekaqdlalsl aqtkasvssl qevamflqas vlerdseqqr lqdeleltrr
2221 alekerlhsp gatstaelgs rgeqgvqlge vsgveaepsp dgmekqswrq rlehlqqava
2281 rleidrsrlq rhnvqlrstl eqvererrkl kreamraaqa gsleiskata ssptqqdgrg
2341 qknsdakcva elqkevvllq aqltlerkqk qdyitrsaqt srelaglhhs lshsllavaq
2401 apeatvleae trrldesltq sltspgpvll hpspsttqaa sr
Mutant keratin 9
      1 mscrqfsssy ltsggggggg lgsggsirss ysrfsssggr ggggrfssss gygggssrvc
    61 grggggsfgy sygggsgggf sasslgggfg ggsrgfggas gggysssggf gggfgggsgg
  121 gfgggygsgf gglggfggga gggdggilta nekstmqeln srlasyldkv qaleeanndl
  181 enkiqdwydk kgpaaiqkny spyyntiddl kdqivdltvg nnktlldidn trmtlddfri
  241 kfemeqnlrq gvdadinglr qvldnltmek sdlemqyetl qeelmalkkn hkeemsqltg
  301 qnsgdvnvei nvapgkdltk tlndmrqeye qliaknrkdi enqyetqitq iehevsssgq
  361 evqssakevt qlrhgvqele ielqsqlskk aaleksledt knrycgqlqm iqeqisnlea
  421 qitdvrqeie cqnqeyslll sikmrlekei etyhnllegg qedfessgag kiglggrggs
  481 ggsygrgsrg gsggsygggg sgggygggsg srggsggsyg ggsgsgggsg ggygggsggg
  541 hsggsggghs ggsggnyggg sgsgggsggg ygggsgsrgg sggshgggsg fggesggsyg
  601 ggeeasgsgg gygggsgkss hs
Kruppel-type  ink finger protein ZNF-70
      1 ihtgerpyqc kecgksfnqs sglsqhrkih tlkkphecdl cgkafchrsh lirhqrihtg
    61 kkpykcdecg kafsqssnli ehrkthtgek p
Similar to RPS2
      1 maddagaagr pggpggpgmg nrggfrggfg sgirgrgrgr grgrgqgrga regkpedkew
    61 mpvtklgrlv kdmkikslee iylfslpike seiidfflgt slkdevlkim pvqkqtragq
  121 rtrfkvfvai gdynghvglg vkcskevata irgalilaki sivpvcrgyw gnkigkphtv
  181 pckvtgrcss vlvrlipapr gtgivsapvp kkllmmagid dcytsargct atlgnfakat
  241 fdaisktysy lppdlwketv ftkslyeeft dhlikthtrv saqrtqapav att
Elongin A2
      1 maagsttlha veklqvrlat ktepkkleky lqklsalpmt adilaetgir ktvkrlrkhq
    61 hvgdfardla arwkklvlvd rntrpgpqdp eesasrqrfg ealqdqekaw gfpenatapr
  121 spshspehrr tarrtppgqq rphprshsre praerkcpri apadsgryra sptrtaplrm
  181 pegpepaapg kqpgrghtha aqggpllcpg cqgqpqgkav vshskghkss rqekrplcaq
  241 gdwhsptlir ekscgaclre etprmpswas ardrqpsdfk tdkeggqags gqrvpaleea
  301 pdshqkrpqh shsnkkrpsl dgrdpgngth glspeekeql sndretqegk pptahldrts
  361 vsslseveev dmaeefeqpt iscekyltyd qlrkqkkktg ksattalgdk qrkaneskgt
  421 reswdsakkl ppvqesqser lqaagadsag pktvpnhvfs elwdlseawm qanydplsds
  481 dsmtsqakpe alsspkfree aafpgrrvna kmpvysgsrp acqlqvptlr qqcaqvlrnn
  541 pdalsdvgev pywvlepvle gwrpdqlyrr kkdnhalvre tdelrrnhcf qdfkeekpqe
  601 nktwreqylr lpdapeqrlr vmttnirsar gnnpngreak micfksvakt pydtsrrqek
  661 sagdadpeng eikpaskpag sshtpssqss sgggrdssss ilrwlpekra npclsssneh
  721 aapaaktrkq aakkvaplma kairdykrrf srr
Tropomyosin alpha
      1 meaikkkmqm lkldkenald raeqaeaeqk qaeerskqle delaamqkkl kgtedeldky
    61 sealkdaqek lelaekkaad aeaevaslnr riqlveeeld raqerlatal qkleeaekaa
  121 desergmkvi enralkdeek meiqeiqike akhiaeeadr kyeevarklv iiegdlerte
  181 eraelaeskc seleeelknv tnnlksleaq aekysqkedk yeeeikiltd klkeaetrae
  241 faersvakle ktiddledel yaqklkykai seeldhalnd mtsi
ATP synthase beta chain (NP_001677)
      1 mlgfvgrvaa apasgalrrl tpsaslppaq lllraaptav hpvrdyaaqt spspkagaat
    61 grivavigav vdvqfdeglp pilnalevqg retrivieva qhlgestvrt iamdgteglv
  121 rgqkvldsga pikipvgpet lgrimnvige pidergpikt kqfapihaea pefmemsveq
  181 eilvtgikvv dllapyakgg kiglfggagv gktvlimeli nnvakahggy svfagvgert
  241 regndlyhem iesgvinlkd atskvalvyg qmneppgara rvaltgltva eyfrdqegqd
  301 vllfidnifr ftqagsevsa llgripsavg yqptlatdmg tmqeritttk kgsitsvqai
  361 yvpaddltdp apattfahld attvlsraia elgiypavdp ldstsrimdp nivgsehydv
  421 argvqkilqd ykslqdiiai lgmdelseed kltvsrarki qrflsqpfqv aevftghmgk
  481 lvplketikg fqqilageyd hlpeqafymv gpieeavaka dklaeehss
Small ribosomal protein 4
      1 PGLTNKTSQL KSSSINQSTS NKKISQYRIR LEEKQKLRFH YGIIERQLLN YVRIARKAKG
    61 STGEVLLQLL EMRSDNVIFR LGMAPTIPGA RQLVNHRHIL VNDRIVDIPS YRCKPQDFIT
  121 IKNRQKSQAI INKNIDFYQK YKIPNHLTYS SLEKKGFVNQ ILDRESIGLK INELLVVEYY
  181 SRQA
Nebulin
      1 maddedyeev veyyteevvy eevpgetitk iyettttrts dyeqsetskp alaqpalaqp
    61 asakpverrk virkkvdpsk fmtpyiahsq kmqdlfspnk ykekfektkg qpyasttdtp
  121 elrrikkvqd qlsevkyrmd gdvaktichv dekakdieha kkvsqqvskv lykqnwedtk
  181 dkyllppdap elvqavknta mfskklyted weadkslfyp yndspelrrv aqaqkalsdv
  241 aykkglaeqq aqftpladpp diefakkvtn qvskqkyked yenkikgkws etpcfevana
  301 rmnadnistr kyqedfenmk dqiyfmqtet peykmnkkag vaaskvkyke dyeknkgkad
  361 ynvlpasenp qlrqlkaagd alsdklyken yektkaksin ycetpkfkld tvlqnfssdk
  421 kykdsylkdi lghyvgsfed pyhshcmkvt aqnsdknyka eyeedrgkgf fpqtitqeye
  481 aikkldqckd htykvhpdkt kftqvtdspv llqaqvnskq lsdlnykakh esekfkchip
  541 pdtpafiqhk vnaynlsdnl ykqdwekska kkfdikvdai pllaakantk ntsdvmykkd
  601 yeknkgkmig vlsinddpkm lhslkvaknq sdrlykenye ktkaksmnyc etpkyqldtq
  661 lknfsearyk dlyvkdvlgh yvgsmedpyh thcmkvaaqn sdksykaeye edkgkcyfpq
  721 titqeydaik kldqckdhty kvhpdktkft avtdspvllq aqlntkqlsd lnykakhege
  781 rfkchipada pqfiqhrvna ynlsdnvykq dwekskakkf dikvdaipll aakantknts
  841 dvmykkdyek skgkmigals inddpkmlhs lktaknqsdr eyrkdyeksk tiytapldml
  901 qvtqakksqa iasdvdykhi lhsysyppds invdlakkay alqsdveyka dynswmkgcg
  961 wvpfgsleme kakrasdiln ekkyrqhpdt lkftsiedap itvqskinqa qrsdiaykak
1021 geeiihnynl ppdlpqfiqa kvnaynisen mykadlkdls kkgydlrtda ipiraakaar
1081 qaasdvqykk dyekakgkmv gfqslqddpk lvhymnvaki qsdreykkdy ektkskyntp
1141 hdmfnvvaak kaqdvvsnvn ykhslhhyty lpdamdlels knmmqiqsdn vykedynnwm
1201 kgigwipigs ldvekvkkag dalnekkyrq hpdtlkftsi vdspvmvqak qntkqvsdil
1261 ykakgedvkh kytmspdlpq flqakcnays isdvcykrdw hdlirkgnnv lgdaipitaa
1321 kasrniasdy kykeayeksk gkhvgfrslq ddpklvhymn vaklqsdrey kknyentkts
1381 yhtpgdmvti taakmaqdva tnvnykqplh hytylpdams lehtrnvnqi qsdnvykdey
1441 nsflkgigwi pigslevekv kkagdalner kyrqhpdtvk ftsvpdsmgm mlaqhntkql
1501 sdlnykvege klkhkytidp elpqfiqakv nalnmsdahy kadwkktirk gydlrpdaip
1561 ivaakssrni asdckykeay ekakgkqvgf lslqddpklv hymnvakiqs dreykkgyea
1621 sktkyhtpld mvsvtaakks qevatnanyr qsyhhytllp dalnvehsrn amqiqsdnly
1681 ksdftnwmkg igwvpiesle vekakkagei lsekkyrqhp eklkftyamd tmeqalnksn
1741 klnmdkrlyt ekwnkdktti hvmpdtpdil lsrvnqitms dklykagwee ekkkgydlrp
1801 daiaikaara srdiasdyky kkayeqakgk higfrsledd pklvhfmqva kmqsdreykk
1861 gyeksktsfh tpvdmlsvva akksqevatn anyrnvihty nmlpdamsfe laknmmqiqs
1921 dnqykadyad fmkgigwlpl gsleaeknkk ameiisekky rqhpdtlkys tlmdsmnmvl
1981 aqnnakimne hlykqawead ktkvhimpdi pqiilakana inisdklykl sleeskkkgy
2041 dlrpdaipik aakasrdias dykykynyek gkgkmvgfrs leddpklvhs mqvakmqsdr
2101 eykknyentk tsyhtpadml svtaakdaqa nitntnykhl ihkyillpda mnieltrnmn
2161 riqsdneykq dynewykglg wspagsleve kakkateyas dqkyrqhpsn fqfkkltdsm
2221 dmvlakqnah tmnkhlytid wnkdktkihv mpdtpdilqa kqnqtlysqk lyklgweeal
2281 kkgydlpvda isvqlakasr diasdykykq gyrkqlghhv gfrslqddpk lvlsmnvakm
2341 qsereykkdf ekwktkfssp vdmlgvvlak kcqelvsdvd yknylhqwtc lpdqndvvqa
2401 kkvyelqsen lyksdlewlr gigwsplgsl eaeknkrase iisekkyrqp pdrnkftsip
2461 damdivlakt naknrsdrly reawdkdktq ihimpdtpdi viakanlint sdklyrmgye
2521 elkrkgydlp vdaipikaak asreiaseyk ykegfrkqlg hhigarnied dpkmmwsmhv
2581 akiqsdreyk kdfekwktkf sspvdmlgvv laykcqtlvs dvdyknylhq wtclpdqsdv
2641 iharqaydlq sdnlyksdlq wlkgigwmts gsledeknkr atqilsdhvy rqhpdqfkfs
2701 slmdsipmvl aknnaitmnh rlyteawdkd kttvhimpdt pevllakqnk vnyseklykl
2761 gleeakrkgy dmrvdaipik aakasrdias efkykegyrk qlghhigara irddpkmmws
2821 mhvakiqsdr eykkdfekwk tkfsspvdml gvvlakkcqt lvsdvdykny lhqwtclpdq
2881 sdviharqay dlqsdnmyks dlqwmrgigw vsigsldvek ckrateilsd kiyrqppdrf
2941 kftsvtdsle qvlaknnaln mnkrlyteaw dkdktqihim pdtpeimlar qnkinysetl
3001 yklaneeakk kgydlrsdai pivaakasrd visdykykdg yrkqlghhig arnieddpkm
3061 mwsmhvakiq sdreykkdfe kwktkfsspv dmlgvvlakk cqtlvsdvdy knylhewtcl
3121 pdqndvihar qaydlqsdni yksdlqwlrg igwvpigsmd vvkckraaei lsdniyrqpp
3181 dklkftsvtd sleqvlaknn alnmnkrlyt eawdkdktqv himpdtpeim larqnkinys
3241 eslyrqamee akkegydlrs daipivaaka srdiasdyky keayrkqlgh higaravhdd
3301 pkimwslhia kvqsdreykk dfekyktrys spvdmlgivl akkcqtlvsd vdykhplhec
3361 iclpdqndii harkaydlqs dnlyksdlew mkgigwvpid slevvrakra gellsdtiyr
3421 qrpetlkfts itdtpeqvla knnalnmnkr lyteawdndk ktihvmpdtp eimlaklnri
3481 nysdklykla leeskkegyd iridaipiqa akasrdiasd ykykegyrkq lghhigarni
3541 kddpkmmwsi hvakiqsdre ykkefekwkt kfsspvdmlg vvlakkcqil vsdidykhpl
3601 hewtclpdqn dviqarkayd lqsdaiyksd lewlrgigwv pigsvevekv krageilsdr
3661 kyrqpadqlk ftcitdtpei vlaknnaltm skhlyteawd adktsihvmp dtpdillaks
3721 nsanisqkly tkgwdeskmk dydiradais iksakasrdi asdykykeay ekqkghhiga
3781 qsieddpkim caihaekiqs ereykkefqk wktkfsspvd mlsillakkc qtlvtdiyyr
3841 nylhewtcmp dqndiiqakk aydlqsdaly kadlewirgi gwmpqgspev lrvknaqnif
3901 cdsvyrtpvv nlkytsivdt pevvlaksna enisipkyre vwdkdktsih impdtpeinl
3961 aranalnvsn klyregwdem kagcdvrlda ipiqaakasr eiasdykykl dhekqkghyv
4021 gtltarddnk irwaliadkl qnereyrldw akwkakiqsp vdmlsilhsk nsqalvsdmd
4081 yrnylhqwtc mpdqndviqa kkayelqsdn vykadlewir gigwmpndsv svnhakhaad
4141 ifsekkyrtk ietlnftpvd drvdyvtakq sgeilddiky rkdwnatksk ytltetpllh
4201 taqeaarild qylykegwer qkatgyilpp davpfvhahh cndvqselky kaehvkqkgh
4261 yvgvptmrdd pklvwfehag qiqnerlyke dyhktkakin ipadmvsvla akqgqtlvsd
4321 idyrnylhqw mchpdqndvi qarkaydlqs dnvyradlew lrgigwipld svdhvrvtkn
4381 qemmsqikyk knalenypnf tsvvdppeiv lakinsvnqs dvkyketfnk akgkytfspd
4441 tphishskdm gklystilyk gawegtkayg ytlderyipi vgakhadlvn selkyketye
4501 kqkghylagk vigefpgvvh cldfqkmrsa lnyrkhyedt kanvhipndm mnhvlakrcq
4561 yilsdleyrh yfhqwtslle epnvirvrna qeilsdnvyk ddlnwlkgig cyvwdtpqil
4621 hakksydlqs qlqytaagke nlqnynlvtd tplyvtavqs ginasevkyk enyhqikdky
4681 ttvletvdyd rtrnlknlys snlykeawdr vkatsyilps stlslthakn qkhlashiky
4741 reeyekfkal ytlprsvddd pntarclrvg klnidrlyrs vyeknkmkih ivpdmvemvt
4801 akdsqkkvse idyririhew ichpdlqvnd hvrkvtdqis divykddlnw lkgigcyvwd
4861 tpeilhakha ydlrddikyk ahmlktrndy klvtdtpvyv qavksgkqls davyhydyvh
4921 svrgkvaptt ktvdldralh ayklqssnly ktslrtlptg yrlpgdtphf khikdtryms
4981 syfkykeaye htkaygytlg pkdvpfvhvr rvnnvtserl yrelyhklkd kihttpdppe
5041 irqvkktqea vseliyksdf fkmqghmisl pytpqvihcr yvgditsdik ykedlqvlkg
5101 fgcflydtpd mvrsrhlrkl wsnylytdka remrdkykvv ldtpeyrkvq elkthlselv
5161 yraagkkqks iftsvpdtpd llrakrgqkl qsqylyvela tkerphhhag nqttalkhak
5221 dvkdmvsekk ykiqyekmkd kytpvpdtpi lirakraywn asdlryketf qktkgkyhtv
5281 kdaldivyhr kvtddiskik ykenymsqlg iwrsipdrpe hfhhravtdt vsdvkykedl
5341 twlkgigcya ydtpdftlae knktlyskyk ykevfertks dfkyvadspi nrhfkyatql
5401 mnekkyrady eqrkdkyhlv vdeprhllak trsdqisqik yrknyekskd kftsivdtpe
5461 hlrttkvnkq isdilykley nkakprgytt ihdtpmllhv rkvkdevsdl kykevyqrnk
5521 snctiepdav hikaakdayk vntnldykkq yeankahwkw tpdrpdflqa aksslqqsdf
5581 eykldreflk gcklsvtddk ntvlalrntl iesdlkykek hvkergtcha vpdtpqilla
5641 ktvsnlvsen kykdhvkkhl aqgsyttlpe trdtvhvkev tkhvsdtnyk kkfvkekgks
5701 nysimleppe vkhamevakk qsdvayrkda kenlhyttva drpdikkatq aakqasevey
5761 rakhrkegsh glsmlgrpdi emakkaakls sqvkyrenfd kekgktpkyn pkdsqlykvm
5821 kdannlasev kykadlkklh kpvtdmkesl imnhvlntsq lassyqykkk yekskghyht
5881 ipdnleqlhl keatelqsiv kykekyeker gkpmldfetp tyitakesqq mqsgkeyrkd
5941 yeesikgrnl tglevtpall hvkyatkias ekeyrkdlee sirgkgltem edtpdmlrak
6001 natqilneke ykrdlelevk grglnamane tpdfmrarna tdiasqikyk qsaemekanf
6061 tsvvdtpeii haqqvknlss qkkykedaek smsyyetvld tpeiqrvren qknfsllqyq
6121 cdlknskgki tvvqdtpeil rvkenqknfs svlykedvsp gtaigktpem mrvkqtqdhi
6181 ssvkykeaig qgtpipdlpe vkrvketqkh issvmykenl gtgipttvtp eiervkrnqe
6241 nfssvlyken lgkgiptpit pemervkrnq enfssvlyke nmgkgtplpv tpemervkhn
6301 qenissvlyk envgkatatp vtpemqrvkr nqenissvly kenlgkatpt pftpemervk
6361 rnqenfssvi ykenmrkatp tpvtpemera krnqenissv lysdsfrkqi qgkaayvldt
6421 pemrrvretq rhistvkyhe dfekhkgcft pvvtdpiter vkknmqdfsd inyrgiqrkv
6481 vemeqkrndq dqetitglrv wrtnpgsvfd ydpaedniqs rslhminvqa qrrsreqsrs
6541 asalsvsgge eksehseapd hhlstysdgg vfavstaykh akttelpqqr sssvatqqtt
6601 vssipshpst agkiframyd ymaadadevs fkdgdaiinv qaidegwmyg tvqrtgrtgm
6661 lpanyveai
Similar to serine protease
      1 mpssvswgil llaglcclvp vslaedpqgd aaqktdtshh dqdhptfnki tpnlaefafs
    61 lyrqlahqsn stniffspvs jatafamisi gtkadthdei leglnfnlte ipeaqihegf
  121 qellrtlnqp dsqlqlttgn gifisegiki vdkfledvkk lyhseaftvn fgdteeakkq
  181 indyvekgtq gkivdlvkel drdtvfalvn yiffkgkwer pfevkdteee dfhvdqvttv
  241 kvpmmkrlgm fniqhckkls swvllmkylg nataifflpd egklqhlene lthdiitkfl
  301 enedrrsasl hlpklsitgt ydlksvlgql gitkvfsnga dlsgvteeap lklskavhka
  361 vltidekgte aagamfleai pmsippevkf nkpfvflmie qntksplfmg kvvnptqk
Complement component Clr
      1 mwllyllvpa lfcraggsip ipqklfgevt splfpkpypn nfetttvitv ptgyrvklvf
    61 qqfdlepseg cfydyvkisa dkkslgrfcg qlgsplgnpp gkkefmsqgn kmlltfhtdf
  121 sneengtimf ykgflayyqa vdldecasrs ksgeedpqpq cqhlchnyvg gyfcscrpgy
  181 elqedrhscq aecsselyte asgyissley prsyppdlrc nysirvergi tlhlkflepf
  241 diddhqqvhc pydqlqiyan gknigefcgk qrppdldtss navdllfftd esgdsrgwkl
  301 rytteiikcp qpktldefti iqnlqpqyqf rdyfiatckq gyqliegnqv lhsftavcqd
  361 dgtwhrampr ckikdcgqpr nlpngdfryt ttmgvntyka riqyychepy ykmqtragsr
  421 eseqgvytct aqgiwkneqk gekiprclpv cgkpvnpveq rqriiggqka kmgnfpwqvf
  481 tnihgrggga llgdrwilta ahtlypkehe aqsnasldvf lghtnveelm klgnhpirrv
  541 svhpdyrqde synfegdial lelensvtlg pnllpiclpd ndtfydlglm gyvsgfgvme
  601 ekiahdlrfv rlpvanpqac enwlrgknrm dvfsqnmfca ghpslkqdac qgdsggvfav
  661 rdpntdrwva tgivswgigc srgygfytkv lnyvdwikke meeed
K1AA1273/TOB3 = K1AA1273/TOB3
      1 swvqmqleal nllhtlvwar slcragavqt qerlsgsasp eqvpagecca lqeyeaaveq
    61 lkseqiraqa eerrktlsee trqhqaraqy qdklarqrye dqlkqqqlln eenlrkqees
  121 vqkqeamrra tveremelrh knemlrvete ararakaere nadlireqir lkasehrqtv
  181 lesirtagtl fgegfrafvt drdkvtatva gltllavgvy saknatavtg rfiearlgkp
  241 slvretsrit vlealrhpiq vsrrllsrpq dvlegvvlsp slearvrdia iatrntkknr
  301 glyrhillyg ppgtgktlfa kklalhsgmd yaimtggdva pmgregvtam hklfdwants
  361 rrglllfmde adaflrkrat eeiskdlrat lnaflyhmgq hsnkfmlvla snlpeqfdca
  421 insridvmvh fdlpqqeere rlvrlhfdnc vlkpategkr rlklaqfdyg rkcsevarlt
  481 egmsgreiaq lavswqatay askdgvltea mmdacvqdav qqyrqkmrwl kaegpgrgve
  541 hplsgvqget ltswslatdp sypclagpct fricswmgtg lcpgplsprm scgggrpfcp
  601 pghpll
K1AA1937
VLGGLLLLGV AQLGLFLEEK LREHLAEKEK LNEERLEQEE KLKAKIRQLT EEKAALEEYI
TQERNRAKET LEEERKRMQE LESLLAQQKK ALAKSITQEK NRVKEALEEE QTRVQELEER
LARQKEISES NIAYEKRKAK EAMEKEKKKV QDLENRLTKQ KEELELKEQK EDVLNNKLSD
ALAMVEETQK TKATESLKAE SLALKLNETL AELETTKTKN IMVEERLILQ QKMVKALQDE
QESQRHGFGE EIMEYKEQIK QHAQTIVSLE EKLQKVTQHH KKIEGEIATL KDNDPAPKEE
RPQDPLVAPM TESSAKDMAY EHLIDDLLAA QKEILSQQEV IMKLRKDLTE AHSRMSDLRG
ELNEKQKMEL EQNVVLVQQQ SKELSVLKEK MAQMSSLVEK KDRELKALEE ALRASQEKHR
LQLNTEKEQK PRKKTQTCDT SVQIEPVHTE AFSSSQEQQS FSDLGVRCKG SRHEEVIQRQ
KKALSELRAR IKELEKARSP DHKDHQNESF LDLKNLRMEN NVQKILLDAK PDLPTLSRIE
ILAPQNGLCN ARFGSAMEKS GKMDVAEALE LSEKLYLDMS KTLGSLMNIK NMSGHVSMKY
LSRQEREKVN QLRQRDLDLV FDKITQLKNQ LGRKEELLRG YEKDVEQLRR SKVSIEMYQS
QVAKLEDDIY KEAEEKALLK EALERMEHQL CQEKRINRAI RQQK
KIAA1000
LYLPHLTKKI MVESCLLTFR AFFWWIALIK MDLSDLGEAA AFLRRSEAEL LLLQATALDG
KKKCWIPDGE NAYIEAEVKG SEDDGTVIVE TADGESLSIK EDKIQQMNPP EFEMIEDMAN
LTHLNEASVL HTLKRRYGQW MIYTYSGLFC VTINPYKWLP VYQKEVMAAY KGKRRSEAPF
HIFAVANNAF QDMLHNRENQ SILFTGESGA GKTVNSKHII QYFATIAAMI ESRKKQGALE
DQIMQANTTL EAFGNAKTLR NDNSSRFGKF IRMHFGARGM LSSVDIDIYL LEKSRVIFQQ
AGERNYHIFY QILSGQKELH DLLLVSANPS DFHFCSCGAV TVESLDDAEE LLATEQAMDI
LGFLPDEKYG CYKLTGAIMH FGNMKFKQKP REEQLEADGT ENADKAAFLM GINSSELVKC
LIHPRIKVGN EYVTRGQTIE QVTCAVGALS KSMYERMFKW LVARINRALD AKLSRQFFIG
ILDITGFEIL EYNSLEQLCI NFTNEKLQQF FNWYMFVLEQ EEYKKESIEW VSIGFGLDLQ
ACIDLIEKPM GILSILEEEC MFPKATDLTF KTKLFDNHFG KSVHLQKPKP DKKKFEAHFE
LVHYAGVVPY NISGWLEKNK DLLNETVVAV FQKSSNRLLA SLFENYMSTD SAIPFGEKKR
KKGASFQTVA SLHKENLNKL MTNLKSTAPH FVRCINPNVN KIFGILDPYL VLQQLRCNGV
LEGTRICREG FPNRLQYADF KQRYCILNFR TFPKSKFVSS RKAAEELLGS LEIDHTQYRF
GITKVFFKAG FLGQLEAIRD ERLSKVFTLF QARAQGKLMR IKFQKILEER DALILIQWNI
RAFMAVKNWP WMRLFFKIKP LVKSSEVGEE VAGLKEECAQ LQKALEKSEF QREELKAKQV
SLTQEKNDLI LQLQAEQETL ANVEEQCEWL IKSKIQLEAR VKELSERVEE EEEINSELTA
RGRKLEDECF ELKKEIDDLE TMLVKSEKEK RTTEHKVKNL TEEVEELNED ISKLNRAAKV
VQEAHQQTLD DLHMEEEKLS SLSKANLKLE QQVDELEGAL EQERKARMNC ERELHKLEGN
LKLNRESMEN LESSQRHLAE ELRKKELELS QMNSKVENEK GLVAQLQKTV KELQTQIKDL
KEKLEAERTT RAKMERERAD LTQDLADLNE RLEEVGGSSL AQLEITKKQE TKIQKLHRDM
EEATLHFETT SASLKKRHAD SLAELEGQVE NLQQVKQKLE KDKSDLQLEV DDLLTRVEQM
TRAKANAEKL CTLYEERLHE ATAKLDKVTQ LANDLAAQKT KLWSESGEFL RRLEEKEALI
NQLSREKSNF TRQIEDLRGQ LEKETKSQSA LAHALQKAQR DCDLLREQYE EEQEVKAELH
RTLSKVNAEM VQWRMKYENN VIQRTEDLED AKKELAIRLQ EAAEANGVAN ARNASLEPAR
HQLQLELGDA LSDLGKVRSA AARLDQKQLQ SGKALADWKQ KHEESQALLD ASQKEVQALS
TELLKLKNTY EESIVGQETL RRENKNLQEE ISNLTNQVRE GTKNLTEMEK VKKLIEEEKT
EVQVTLEETE GALERNESKI LHFQLELLEA KAELERKLSE KDEEIENFRR KQQCTIDSLQ
SSLDSEAKSR IEVTRLKKKM EEDLNEMELQ LSCANRQVSE ATKSLGQLQI QIKDLQMQLD
DSTQLNSDLK EQVAVAERRN SLLQSELEDL RSLQEQTERG RRLSEEELLE ATERINLFYT
QNTSLLSQKK KLEADVARMQ KEAEEVVQEC QNAEEKAKKA AIEAANLSEE LKKKQDTIAH
LERTRENMEQ TITDLQKRLA EAEQMALMGS RKQIQKLESR VRELEGELEG EIRRSAEAQR
GARRLERCIK ELTYQAEEDK KNLSRMQTQM DKLQLKVQNY KQQVEVAETQ ANQYLSKYKK
QQHELNEVKE RAEVAESQVN KLKIKAREFG KKVQEE
KIAA1217
KTALLLHGVF LLLLIFFLEQ GKGNLHVTSP EDAECRRTKE RLSNGNSRGS VSKSSRNIPR
RHTLGGPRSS KEILGMQTSE MDRKREAFLE HLKQKYPHHA SAIMGHQERL RDQTRSPKLS
HSPQPPSLGD PVEHLSETSA DSLEANSEGD APTPFSRGSR TRASLPVVRS TNQTKERSLG
VLYLQYGDET KQLRMPNEIT SADTIPALFV SAFPQQLTMK MLESPSVAIY IKDESRNVYY
ELNDVRNIQD RSLLKVYNKD PAHAFNHTPK TMNGDMRMQR ELVYARGDGP GAPRPGSTAH
PPHAIPNSPP STPVPHSMPP SPSRIPYGGT RSMVVPGNAT IPRDRISSLP VSRPISPSPS
AILERRDVKP DEDMSGKNIA MYRNEGFYAD PYLYHEGRMS IASSHGGHPL DVPDHIIAYH
RTAIRSASAY CNPSMQAEMH MEQSLYRQKS RKYPDSHLPT LGSKTPPASP HRVSDLRMID
MHAHYNAHGP PHTMQPDPAS PSRQAFKKEP GTLVYIEKPR SAAGLSSLVD LGPPLMEKQV
FAYSTATIPK DRETRERMQA MEKQIASLTG LVQSALFKGP ITSYSKDASS EKMMKTTANR
NHTDSAGTPH VSGGKMLSAL ESTVPPSQPP PVGTSAIHMS LLEMRRSVAE LRLQLQQMRQ
LQLQNQELLR AMMKKAELEI SGKVMETMKR LEDPVQRQRV LVEQERQKYL HEEEKIVKKL
CELEDEVEDL KKDSTAASRL VTLKDVEDGA FLLRQVGEAV ATLKGEFPTL QNKMPAILRI
EVEAVRFLKE EPHKLDSLLK RVRSMTDVLT MLRRHVTDGL LKGTDAAQAA QYMAMEKATA
AEVLKSQEEA AHTSGQPFHS TGAPGDAKSE VVPLSGMMVR HAQSSPVVIQ PSQHSVALLN
PAQNLPHVAS SPAVPQEATS TLQMSQAPQS PQIPMNGSAM QSLFIEEIHS VSAKNRAVSI
EKAEKKWEEK RQNLDHYNGK EFEKLLEEAQ ANIMKSIPNL EMPPATGPLP RGDAPVDKVE
LSEDSPNSEQ DLEKLGGKSF PPPPPPPRRS YLPGSGLTTT RSGDVVYTGR KENITAKASS
EDAGPSPQTR ATKYPAEEPA SAWTPSPPPV TTSSSKDEEE EEEEGDKIMA ELQGSSGAPQ
TSRMPVPMSA KNRPGTLDKP GKQSKLQDPR QYRQANGSAK KSGGDFKPTS PELPASKIPA
LSPSSGKSSS LPSSSGDSSN LPNPPATKPS IASNPLSPQT GPPAHSASLI PSVSNGSLKF
QSLTHTGKGH HLSFSPQSQN GRAPPPLSFS SSPPSPASSV SLNQGAKGTR TIHTPSLTSY
KAQNGSSSKA TPSTAKETS
cDNA FLJ25393
MTQNYEGQLQ SLKAQFSKLT NNFEKLRLHQ MKQNKVPRKE LPHLKEEIPF ELSNLNQKLE
EFRAKSREWD KQEILYQTHL ISLDAQQKLL SEKCNQFQKQ AQSYQTQLNG KKQCLEDSSS
EIPRLICDPD PNCEINERDE FIIEKLKSAV NEIALSRNKL QDENQKLLQE LKMYQRQCQA
MEAGLSEVKS ELQSRDDLLR IIEMERLQLH RELLKIGECQ NAQGNKTRLE SSYLPSIKEP
ERKIKELFSV MQDQPNHEKE LNKIRSQLQQ VEEYHNSEQE RMRNEISDLT EELHQKEITI
ATVTKKAALL EKQLKMELEI KEKMLAKQKV SDMKYKAVRT ENTHLKGMMG DLDPGRYMSM
DFTNREQSRH TSINKLQYEN ERLRNDLAKL HVNGKSTWTN QNTYEETGRY AYQSQIKVEK
NEERLSHDCE PNRSTMPPLP PSTFQAKEMT SPLVSDDDVF PLSPPDMSFP ASLAAQHFLL
EEEKRAKELE KLLNTHIDEL QRHTEFTLNK YSKLKQNRHI
Unknown protein
HEGPRRRGRL WDSSGVPQRQ KRPGPWRTQT QEQMSRDVCI HTWPCTYYLE PKRRWVTGQL
SLTSLSLRFM TDSTGEILVS FPLSSIVEIK KEASHFIFSS ITILEKGHAK HWFSSLRPSR
NVVFSIIEHF WRELLLSQPG AMADASVPRT RGEELTGLMA GSQKRLEDTA RVLHHQGQQL
DSVMRGLDKM ESDLEVADRL LTELESPAWW PFSSKLWKTP PETKPREDVS MTSCEPFGKE
GILIKIPAVI SHRTESHVKP GRLTVLVSGL EIHDSSSLLM HRFEREDVDD IKVHSPYEIS
IRQRFIGKPD MAYRLISAKM PEVIPILEVQ FSKKMELLED ALVLRSARTS SPAEKSCSVW
HAASGLMGRT LHREPPAGDQ EGTALHLQTS LPALSEADTQ ELTQILRRMK GLALEAESEL
ERQDEALDGV AAAVDRATLT IDKHNRRMKR LT
Hyp. 41.3kD protein
MVLGLTEMLR SCLLYALRPH CCHCHRMPFS IRGHL
Cytidine deaxninase
      1 maqkrpactl kpecvqqllv csqeakksay cpyshfpvga alltqegrif kgcnienacy
    61 plgicaerta iqkavsegyk dfraiaiasd mqddfispcg acrqvmrefg tnwpvymtkp
  121 dgtyivmtvq ellpssfgpe dlqktq
Growth factor receptor-bound protein 2
      1 meaiakydfk ataddelsfk rgdilkvlne ecdqnwykae lngkdgfipk nyiemkphpw
    61 ffgkipraka eemlskqrhd gaflireses apgdfslsvk fgndvqhfkv lrdgagkyfl
  121 wvvkfnslne lvdyhrstsv srnqqiflrd ieqvpqqpty vqalfdfdpq edgelgfrrg
  181 dfihvmdnsd pnwwkgachg qtgmfprnyv tpvnrnv
TGF-beta receptor interacting protein 1
      1 mkpillqghe rsitqikynr egdllftvak dpivnvwysv ngerlgtymg htgavwcvda
    61 dwdtkhvltg sadnscrlwd cetgkqlall ktnsavrtcg fdfggriimf stdkqmgyqc
  121 fvsffdlrdp sqidnnepym kipcndskit savwgplgec jiaghesgel nqysaksgev
  181 lvnvkehsrq indiqlsrdm tmfvtaskdn taklfdsttl ehqktfrter pvnsaalspn
  241 ydhvvlgggq eamdvtttst rigkfearff hlafeeefgr vkghfgpins vafhpdgksy
  301 ssggedgyvr ihyfdpqyfe fefea
Paraspekie protein 1 alpha isoform
MMLRGNLKQV RIEKNPARLR ALESAVGESE PAAAAAMALA LAGEPAPPAP APPEDHPDEE
MGFTIDIKSF LKPGEKTYTQ RCRLFVGNLP TDITEEDFKR LFERYGEPSE VFINRDRGFG
FIRLESRTLA EIAKAELDGT ILKSRPLRIR FATHGAALTV KNLSPVVSNE LLEQAFSQFG
PVEKAVVVVD DRGRATGKGF VEFAAKPPAR KALERCGDGA FLLTTTPRPV IVEPMEQFDD
EDGLPEKLMQ KTQQYHKERE QPPRFAQPGT FEFEYASRWK ALDEMEKQQR EQVDRNIREA
KEKLEAEMEA ARHEHQLMLM RQDLMRRQEE LRRLEELRNQ ELQKRKQIQL RHEEEHRRRE
EEMIRHREQE ELRRQQEGFK PNYMENREQE MRMGDMGPRG AINMGDAFSP APAGNQGPPP
MMGMNMNNRA TIPGPPMGPG PAMGPEGAAN MGTPMMPDNG AVHNDRFPQG PPSQMGSPMG
SRTGSETPQA PMSGVGPVSG GPGGFGRGSQ GGNFEGPNKR RRY
Heterogenous nuclear ribonucleoprotein H
      1 mmlgteggeg fvvkvrglpw scsadevqrf fsdckiqnga qgirfiytre grpsgeafve
    61 lesedevkla lkkdretmgh ryvevfksnn vemdwvlkht gpnspdtand gfvrlrglpf
  121 gcskeeivqf fsgleivpng itlpvdfqgr stgeafvqfa sqeiaekalk khkerighry
  181 ieifkssrae vrthydpprk lmamqrpgpy drpgagrgyn sigrgagfer mrrgaygggy
  241 ggyddyngyn dgygfgsdrf grdlnycfsg msdhrygdgg stfqsttghc vhmrglpyra
  301 tendiynffs plnpvrvhie igpdgrvtge advefathed avaamskdka nmqhryvelf
  361 lnstagasgg ayehryvelf lnstagasgg aygsqmmggm glsnqssygg pasqqlsggy
  421 gggyggqssm sgydqvlqen ssdfqsnia
Profilin II
      1 magwqsyvdn lmcdgccqea aivgycdaky vwaataggvf qsitpieidm ivgkdregff
    61 tngltlgakk csvirdslyv dgdctmdirt ksqggeptyn vavgragrvl vfvmgkegvh
  121 ggglnkkays makylrdsgf
```

### REFERENCES

1. Skoldberg, F., Ronnblom, L., Thornemo, M., Lindahl, A., Bird, P. I., Rorsman, F., Kampe, O. & Landgren, E. (2002) Biochem Biophys Res Cominun 291, 951-8.
2. Ohguro, H., Ogawa, K., Maeda, T., Maeda, A. & Maruyama, I. (1999) Invest Ophthalinol Vis Sci 40, 3160-7.
3. Xue, C., Takahashi, M., Hasunuma, T., Aono, H., Yamamoto, K., Yoshino, S., Sumida, T. & Nishioka, K. (1997) Ann Rheum Dis 56, 262-7.
4. Mack, G. J., Rees, J., Sandblom, O., Balczon, R., Fritzler, M. J. & Rattner, J. B. (1998) Arthritis Rheum 41, 551-8.
5. Kobayashi, S., Tanaka, T., Matsuyoshi, N. & Imamura, S. (1996) FEBS Lett 386, 149-55.
6. Aubry, M., Marineau, C., Zhang, F. R., Zahed, L., Figlewicz, D., Delattre, O., Thomas, G., de Jong, P. J., Julien, J. P. & Rouleau, G. A. (1992) genomeics 13, 641-8.
7. Aso, T., Yamazaki, K., Amimoto, K., Kuroiwa, A., Higashi, H., Matsuda, Y., Kitajima, S. & Hatakeyama, M. (2000) J Biol Chem 275, 6546-52.
8. Perry, S. V. (2001) J Muscle Res Cell Motil 22, 5-49.
9. Leyva, J. A. (2003) Mol Membr Biol 20, 27-33.
10. Yoshihama, M., Uechi, T., Asakawa, S., Kawasaki, K., Kato, S., Higa, S., Maeda, N., Minoshima, S., Tanaka, T., Shimizu, N. & Kenmochi, N. (2002) genomee Res 12, 379-90.
11. Wang, K., Knipfer, M., Huang, Q. Q., van Heerden, A., Hsu, L. C., Gutierrez, G., Quian, X. L. & Stedman, H. (1996) J Biol Chem 271, 4304-14.
12. Long, G. L., Chandra, T., Woo, S. L., Davie, E. W. & Kurachi, K. (1984) Biochemistry 23, 4828-37.
13. Arlaud, G. J., Gaboriaud, C., Garnier, G., Circolo, A., Thielens, N. M., Budayova-Spano, M., Fontecilla-Camps, J. C. & Volanakis, J. E. (2002) Immunobiology 205, 365-82.
14. Nagase, T., Kikuno, R. & Ohara, O. (2001) DNA Res 8, 319-27.
15. Laliberte, J. & Momparler, R. L. (1994) Cancer Res 54, 5401-7.
16. Tari, A. M. & Lopez-Beresiduesin, G. (2001) Semin Oncol 28, 142-7.
17. Asano, K., Kinzy, T. G., Merrick, W. C. & Hershey, J. W. (1997) J Biol Chem 272, 1101-9.
18. Fox, A. H., Lam, Y. W., Leung, A. K., Lyon, C. E., Andersen, J., Mann, M. & Lamond, A. I. (2002) Curr Biol 12, 13-25.
19. Honore, B., Vorum, H. & Baandrup, U. (1999) FEBS Lett 456, 274-80.
20. Honore, B., Madsen, P., Andersen, A. H. & Leffers, H. (1993) FEBS Lett 330, 151-5.
21. T. A. Hembrough, J. Vasudevan, M. M. Allietta, W. F. Glass, S. L. Gonias, J Cell Sci 108, 1071 (1995).

22. T. A. Hembrough, L. Li, S. L. Gonias, J Biol Chem 271, 25684 (1996).

23. T. A. Hembrough, K. R. Kralovich, L. Li, S. L. Gonias, Biochem J 317, 763 (1996).

24. F. Mahdi, Z. Shariat-Madar, R. F. Todd, C. D. Figueroa, A. H. Schmaier, Biood 97, 2342 (2001).

25. F. Mahdi, Z. S. Madar, C. D. Figueroa, A. H. Schmaier, Blood 99, 3585 (May 15, 2002).

26. M. J. Wells et al., J Biol Chem 272, 28574 (1997).

27. C. L. Riopel, I. Butt, M. B. Omary, Cell Motil Cytoskeleton 26, 77 (1993).

1. An ex vivo method for the generation of a population of autologous or allogenic antigen presenting cells (APCs) capable of inducing an effective immune reaction against an immunogenic squamous epithelial carcinoma antigen comprising a protein according to SEQ ID NO: 1 or variants thereof wherein the variants comprise one or more additions, insertions, substitutions and/or deletions as compared to the corresponding protein of SEQ ID NO: 1, and wherein the immunogenic activity of the variant is essentially equivalent to the activity of the corresponding unmodified protein according to SEQ ID NO: 1 wherein said method comprises the following steps:
a) providing autologous or allogenic APCs;
b) contacting the APCs with an effective amount of a peptide fragment or a protein as defined above under conditions enabling the endocytosis, processing and presentation of the peptide fragments by these APCs, and
c) isolating the APCs presenting the corresponding peptides.

2. An ex vivo method for the preparation of genetically engineered APCs capable of inducing an effective immune reaction against a protein as defined in claim 1 comprising the following steps:
a) providing a nucleic acid encoding a protein as defined in claim 1 or a peptide fragment thereof,
b) transfecting the APCs with the nucleic acid; and
c) selecting APCs presenting the peptide fragments.

3. The method according to claim 2 wherein the nucleic acid in step a) is provided in an expression vector.

4. An APC obtainable by the method according to one or more of the claims 1 to 3.

5. The APC according to claim 4 which is a dendritic cell or a B cell.

6. An ex vivo method for the detection and for the recovery of T cells specific for a protein as defined in claim 1 comprising the following steps:
a) providing mammalian, in particular human, T cells or PBMCs;
b) co-culturing the T cells with an APC according to claim 4 or 5 under conditions enabling an activation of the T cells; and
c) determining the presence of a specific activity of the T cells against an APC according to claim 4 or 5;
d) optionally selecting and culturing/expanding such T cells which in step c) have shown a specificity for an APC according to claim 4 or 5.

7. T cells obtainable by the method according to claim 6.

8. A diagnostic composition comprising a protein as defined in claim 1, an antibody or aptamer directed against one or more epitopes of a protein as defined in claim 1 or a T cell according to claim 7.

9. A pharmaceutical composition comprising a therapeutically effective amount of a protein as defined in claim 1, a nucleic acid encoding said protein, a vector containing said nucleic acid, an antibody or aptamer as defined in claim, 8, an APC according to claim 4 or 5, or a T cell according to claim 7 and a pharmaceutically acceptable carrier.

10. The pharmaceutical composition according to claim 9 which is a vaccine.

11. A pharmaceutical or diagnostic composition according to claim 9 or 10 wherein said antibody is selected from the group consisting of a polyclonal antibody, a monoclonal antibody, a humanised antibody, a chimeric antibody, and a synthetic antibody.

12. The composition according to claim 11 wherein the antibody is bound to a toxic and/or detectable agent.

13. The use of the pharmaceutical composition according to any of the claims 9 to 12 in the diagnosis and therapy of a squamous epithelial carcinoma, in particular a squamous epithelial carcinoma in the otolaryngologic, head, and neck region.
